(19) United States
(12) Patent Application Publication
(10) Pub. No.: US 2019/0086414 A1
(43) Pub. Date: Mar. 21, 2019

(54) THREE-DIMENSIONAL PLATFORM FOR TESTING THERAPEUTIC RESPONSES

(71) Applicant: THE TRUSTEES OF COLUMBIA UNIVERSITY IN THE CITY OF NEW YORK, New York, NY (US)

(72) Inventors: Laura Kaufman, New York, NY (US); Asja Guzman, New York, NY (US)

(21) Appl. No.: 16/194,274

(22) Filed: Nov. 16, 2018

Related U.S. Application Data
(63) Continuation-in-part of application No. PCT/US2017/033031, filed on May 17, 2017.
(60) Provisional application No. 62/339,712, filed on May 20, 2016.
(60) Provisional application No. 62/420,402, filed on Nov. 10, 2016.

(51) Int. Cl. G01N 33/574; G01N 33/50; C12N 5/09; C12M 1/12

(52) U.S. Cl. CPC G01N 33/57415; G01N 33/5011; G01N 33/5091; C12N 2503/02; C12M 25/14; C12N 2533/54; C12N 5/0693

(57) ABSTRACT

An in vitro system is provided for evaluating a therapeutic response to a candidate therapeutic agent. The system includes a multicellular aggregate, a cell-bound layer of basement membrane surrounding the multicellular aggregate, and a three-dimensional (3-D) biopolymer matrix, wherein the multicellular aggregate and the cell-bound layer of basement membrane are disposed within the 3-D biopolymer matrix. Methods of using the system are also provided, including methods of diagnosing and treating a cancer in a subject.

### CROSS-REFERENCE TO RELATED APPLICATIONS

The present application is a continuation in part of PCT international application No. PCT/US2017/033031, filed May 17, 2017, which claims priority to U.S. Provisional Patent Application No. 62/339,712, filed on May 20, 2016, and U.S. Provisional Patent Application No. 62/420,402, filed on Nov. 10, 2016. The entire contents of the aforementioned applications are incorporated by reference as if recited in full herein.

### GOVERNMENT FUNDING

This invention was made with government support under grant no. PESO 1227297, awarded by the National Science Foundation. The government has certain rights in the invention.

### FIELD OF THE INVENTION

The present invention relates to a 3-D platform for testing therapeutic responses, such as in the testing of therapeutic responses of in situ and metastasizing cancers. More specifically, aspects of the present invention relate to an in vitro system for evaluating a therapeutic response to a candidate therapeutic agent, methods of preparing the system, methods for evaluating therapeutic responses using the system, and methods for identifying a candidate therapeutic agent as a candidate anti-cancer drug using the system.

### BACKGROUND OF THE INVENTION

Breast cancer deaths occur primarily from metastatic disease that compromises function of critical organs. In carcinomas (epithelium-derived cancers), the most common type of breast cancer, metastasis requires tumor cells to breach the basement membrane (BM), a subtype of extracellular matrix (ECM) that surrounds the primary tumor, invade collagen I- and fat-rich ECM of the adjacent soft tissue, and intravasate into blood or lymph vessels where they will be transported to distant sites (Valastyan et al 2009). While a complex interplay of genetic and epigenetic changes underlies the multi-step metastatic cascade, dynamic interactions between tumor cells and the ECM are increasingly recognized as a key aspect of metastatic progression (Kumar et al 2009, Lu et al 2012).

The BM is a specialized cell-adherent ECM produced jointly by normal and/or pathological epithelial, endothelial, and stromal cells. It is formed in a multi-step process initiated by cells binding laminin at the cell surface and subsequent accumulation of the non-fibrillar collagen IV at the nascent laminin scaffold. This process leads to a dense sheet-like matrix that under normal circumstances separates the epithelium or endothelium from the adjacent stroma (Kalluri 2003, Yurchenco 2011). BM deposition and turnover are often perturbed in cancers, resulting in matrices that are less crosslinked and thus more accessible to degradation and remodeling (Kalluri 2003, Liotta et al 1980, Martinez-Hernandez et al 1983). Discontinuities of BM surrounding primary tumors are caused by altered expression and crosslinking of BM components as well as enhanced enzymatic degradation, all hallmarks of aggressive cancers and each of established prognostic value (Frei 1962, Bosman et al 1985, Spaderna et al 2006, Bergamaschi et al 2008, Polyak 2010).

In contrast to the non-fibrillar BM, stromal ECM in most organs and connective tissues is dominated by collagen I, a fibrillar collagen (Mouw et al 2014). The stromal ECM also displays abnormalities in composition and organization during carcinogenesis, which lead to changes in biomechanical properties and matrix architecture. The high breast tissue density associated with poor prognosis in patients with breast cancer is due in part to enhanced deposition of mostly fibrillar collagens (Zhu et al 1995, Kauppila et al 1998, Huijbers et al 2010, Alowami et al 2003, Guo et al 2001). Moreover, highly linearized and aligned collagen at tumor boundaries has been found to contribute to tumor invasion and linked to poor prognosis (Provenzano et al 2006, Conklin et al 2011).

At the molecular level, cancer progression and metastasis have long been associated with the epithelial-mesenchymal transition (EMT). This process includes aberrant activation of transcription factors, altered expression and reorganization of cell-surface and cytoskeletal proteins, and production of ECM-degrading enzymes, together resulting in a pro-migratory cellular phenotype (Kalluri et al 2009, Gurzu et al 2015). The contribution of BM/ECM biomechanics to tumor progression has also been recognized, and several studies have reported stiffness-driven induction of EMT (Leight et al 2012, Wei et al 2015) and dramatic changes in invasive behavior in response to matrix stiffness and architecture (Levental et al 2009, Lang et al 2015, Guzman et al 2014).

Still, the cellular processes that lead to and occur alongside tumor cells traversing the BM layer and entering the surrounding ECM as invasive entities are insufficiently understood. This incomplete understanding is caused in part by the considerable difficulties of studying these processes in vivo and in vitro. In vivo, studies are hindered by limitations related to microscopic observations at the tumor site including imaging depth, resolution and overall imaging quality degraded by light scattering and physiological motion (Condeelis et al 2003, Ellenbroek et al 2014). In contrast, while in vitro approaches offer good optical accessibility, they often use models of limited physiological relevance. Such in vitro studies typically rely on either 2D models (Furuyama 2000), which do not recapitulate the dimensionality and biomechanics of the tumor microenvironment, or cells seeded in 3D matrices that do not mimic the tumor architecture or the heterogeneous nature of the tumor environment at the BM/ECM interface.

While studies employing multicellular tumor spheroids (MTSs or spheroids) embedded in biopolymer matrices overcome some of these issues and represent a good model for cancer cell invasion in soft tissue, they do not recapitulate the initial invasive events, namely transmigration of the BM (Thoma et al 2014, Kaufman et al 2005, Guzman et al 2014, Kim et al 2015, Fang et al 2013, Cheung et al 2013). One study used pre-casted basement membrane extract (BME)-cell plugs, which were subsequently embedded into collagen gels (Katz et al 2011). However, in that study there was no spheroid resembling the architecture and biochemical gradients of a solid tumor in vivo, since the plugs are dispersed cells in BME gel. There was also no formation of a basement membrane in that experimental system. Indeed, there are very few studies that address cancer cells consecutively migrating through BM and invading into stromal ECM as occurs in vivo (Katz et al 2011, Schoumacher et al 2010).

### SUMMARY OF THE INVENTION

Aspects of the present invention may bridge the gap in drug testing between rudimentary, poorly predictive 2D systems and physiologically relevant but time-intensive animal studies. Aspects of the present invention may be particularly useful and even critical for testing drugs targeting cell-matrix interactions, such as chemotherapeutics that are anti-invasive rather than anti-proliferative.

Aspects of the in vitro model described herein thus combine multicellular spheroid/organoid with a cell-bound BM and adjacent ECM. In one aspect, the BM is assembled in a cell-catalyzed reaction, which is different from a mere layer of basement membrane extract (BME) hydrogel polymerized in a cell independent reaction. In one aspect, the BM-surrounded cell aggregates are embedded into tunable 3D biopolymer matrices which can be easily supplemented with therapeutic agents and/or cells of the immune system.

According to aspects of the invention, this multi-component system is physiologically highly relevant as it models the solid tumor with the tumor-bound, degradable basement membrane and the adjacent microenvironment, but does not require complex microfluidic approaches or other sophisticated manufacturing techniques. In one embodiment this system is suitable for parallelized processing (96-wells and higher), and combines high physiological relevance with a straightforward workflow that achieves low resource consumption and requires only a short time for experiment execution (24-48 hours).

In one embodiment, an in vitro system for evaluating a therapeutic response to a candidate therapeutic agent is provided. The system includes a multicellular aggregate, a cell-bound layer of basement membrane surrounding the multicellular aggregate, and a three-dimensional (3-D) biopolymer matrix, wherein the multicellular aggregate and the cell-bound layer of basement membrane are disposed within the 3-D biopolymer matrix.

According to yet another embodiment, a method of preparing an in vitro system for evaluating a therapeutic response to a candidate therapeutic agent includes suspending cells in a growth medium supplemented with a basement membrane extract, centrifuging the suspended cells, followed by incubating the cells under conditions sufficient to form a multicellular aggregate surrounded by a layer of basement membrane, and disposing the multicellular aggregate surrounded by the layer of basement membrane in a 3-D extracellular matrix.

According to yet another embodiment, a method for evaluating a therapeutic response to a candidate therapeutic agent in an in vitro system includes providing a candidate therapeutic agent to an in vitro system having a multicellular aggregate, a cell-bound layer of basement membrane surrounding the multicellular aggregate, and a three-dimensional (3-D) biopolymer matrix, wherein the multicellular aggregate and the cell-bound layer of basement membrane are disposed within the 3-D biopolymer matrix, and evaluating the response of cells in the biopolymer matrix to the candidate therapeutic agent.

According to yet another embodiment, a method for identifying a candidate therapeutic agent as a candidate anti-cancer drug, includes contacting a candidate therapeutic agent with an in vitro system having a multicellular aggregate, a cell-bound layer of basement membrane surrounding the multicellular aggregate, and a three-dimensional (3-D) biopolymer matrix, wherein the multicellular aggregate and the cell-bound layer of basement membrane are disposed within the 3-D biopolymer matrix, and evaluating what effect, if any, the candidate therapeutic agent has on the in vitro system, wherein decreased migratory capacities and/or increased cell death of the cells in the multicellular aggregate relative to a control indicates that the candidate therapeutic agent may be a candidate anti-cancer drug.

In another embodiment, a method of treating or ameliorating the effects of a cancer in a subject is provided, which method comprises diagnosing the presence of tumorigenic cells in the subject by the methods of the present invention and administering to the subject an effective amount of an anti-cancer drug.

In another embodiment, a method for diagnosing the presence of tumorigenic cells in a subject is provided, which method comprises obtaining cells from the subject; incubating the cells under conditions sufficient to form a multicellular aggregate surrounded by a layer of basement membrane; disposing the multicellular aggregate surrounded by the layer of basement membrane in a 3-D extracellular matrix; culturing the 3-D extracellular matrix under conditions sufficient to support growth of the cells; and identifying the cells as tumorigenic cells if the multicellular aggregate breaches the layer of basement membrane into the 3-D extracellular matrix.

### BRIEF DESCRIPTION OF THE DRAWINGS

FIG. 1A-FIG. 1C show a schematic representation of the experimental model used in a study according to the present invention. In FIG. 1A, a multicellular tumor spheroid is produced by centrifuging cells with growth media and BM components and incubating for 24 hours under low cell adhesion conditions. Spheroids fully surrounded by a BM layer are then embedded in a biopolymer solution of collagen I and/or BME that undergoes gelation. FIG. 1B shows a schematic depiction of transmigration of cells through the BM layer and invasion into a surrounding collagen I matrix that recapitulates initial steps of invasion in carcinomas. FIG. 1C shows a schematic view of an embodiment of a 3D platform for testing therapeutic response.

FIG. 2A-FIG. 2F show the presence and composition of the basement membrane layer. FIG. 2A-FIG. 2B are confocal fluorescence (CFM) images of a representative MB468 spheroid surrounded by a BM layer at 2 hours (FIG. 2A) and 24 hours (FIG. 2B) post-implantation in a fluorescent bead-loaded collagen I gel. Cells are dyed with Vybrant DiD live cell-labeling solution and false colored in red, while beads appear in white. The BM layer is represented by the area free from bead and cell fluorescence. Its size and integrity change over the course of 24 hours, and beads can be seen within the spheroid at that time point. Scale bar=200 μm. FIG. 2C-FIG. 2D show confocal reflectance (CRM) images of a representative MB468 spheroid surrounded by a BM layer and embedded in a collagen I gel 2 hours (FIG. 2C) and 24 hours (FIG. 2D) after embedding. At 2 hours, the spheroid surface is isolated from the collagen network through the BM layer. The area devoid of collagen fibers (marked by a punctate red line) reflects the thickness of the BM. At 24 hours, cell contact with collagen fibers and radial alignment of these fibers (red arrow), indicating traction generation, is evident. In FIG. 2C-FIG. 2D, image processing software has been used to remove an optical artifact (a bright spot covering ˜9*10³ square pixels in the image) present in confocal reflectance images. Scale bar=50 μm. FIG. 2E shows a representative confocal fluorescence maximum projection constructed from a z-scan over 150 μm of a shelled MB468 spheroid (red) with a BM layer containing fluorescently labeled laminin (green). Distinctive laminin accumulations at the surface of the spheroid are apparent. Scale bar=200 μm. At right, a higher magnification maximum projection over 60 μm of a region of the spheroid is shown. Scale bar=50 μm. FIG. 2F shows a representative confocal fluorescence maximum projection constructed from a z-scan over 18 μm of a shelled MB468 spheroid containing fluorescently labeled type IV collagen (green) shows a dense scaffold fully surrounding the spheroid. Scale bar=200 μm.

FIG. 3A-FIG. 3F show how BM layer thickness varies with exogenous protein concentration. FIG. 3A-FIG. 3D are representative confocal fluorescence images of BM layers around MB468 spheroids generated with addition of 0.002 mg/ml (FIG. 3A), 0.003 mg/ml (FIG. 3B), 0.005 mg/ml (FIG. 3C), and 0.006 mg/ml (FIG. 3D) collagen IV. Images are maximum projections over 30 μm centered at the spheroid midpoint axially. Images show spheroids 1 hour after implantation into collagen. Scale bar=200 pm. FIG. 3E shows how quantification of BM cross-sectional area as a function of exogenous collagen IV concentration demonstrates correlation between these quantities. For this analysis, the area of the spheroid derived from transmitted light images was subtracted from the area of the respective BM shell derived from confocal fluorescence maximum projection. Mean values±SD are shown, sample number n≥7 for every condition. In FIG. 3F, comparative analysis of the area of BM shells as derived from collagen IV fluorescence (as in FIG. 3A-FIG. 3D) and as derived from fluorescent bead exclusion area (as in FIG. 2A) demonstrates that the collagen IV-positive scaffold has a comparable thickness at a given collagen IV concentration to that of the total BM shell. Box plots show first to third quartiles with median denoted by a line and mean with a symbol; whiskers show minimal and maximal values, sample number n≥10 for every condition.

FIG. 4A-FIG. 4C show how non-cancerous MCF10A cells do not breach the BM layer. In FIG. 4A, a representative confocal fluorescence maximum projection of a phalloidin-stained MCF10A MTS 24 hours after embedding in 3D collagen matrix depicts sheet-like expansion and spreading of the spheroid into the matrix. Scale bar=200 μm. Inset in the left corner shows a transmitted light image of this spheroid 1 hour after implantation into collagen. In FIG. 4B, a representative confocal fluorescence maximum projection of an MCF10A spheroid surrounded by a fluorescently labelled BM layer 24 hours after embedding in collagen I shows complete containment of the spheroid within the BM borders. Scale bar=200 μm. Inset shows a transmitted light image of this spheroid 1 hour after implantation into collagen. FIG. 4C shows dependence of MCF10A spheroid invasive area on presence of the BM shell. Invasive area defined as the difference between spheroid area at t=2 hours and t=24 hours for MCF10A spheroids without and with a BM shell are shown in box plots depicting first to third quartiles with median denoted by a line and mean with a symbol; whiskers show minimum and maximum values. n≥9 for each condition.

FIG. 5A-FIG. 5C show how oncogenically transformed MCF10A-Ras cells effectively transmigrate the BM layer. FIG. 5A is a representative confocal fluorescence maximum projection of a BM-shelled phalloidin-stained MCF10A-HRas spheroid 24 hours after embedding in a 3D collagen I matrix, showing a combination of individual and collective invasion of cells into the surroundings. The white square defines a site of collective invasion that is shown at higher magnification in FIG. 5B and FIG. 5C. Cells are shown in red and BM layer is shown in green. Scale bar=200 μm. High magnification confocal fluorescence (FIG. 5B) shows dense packing of cells during collective transmigration of the BM layer and subsequent dissemination as individual cells beyond the BM shell. High magnification confocal reflectance (FIG. 5C) shows extensive collagen reorganization and alignment by the multicellular invasive stream at the interface of the BM and collagen matrix. Scale bar=50 μm.

FIG. 6A-FIG. 6F are images that demonstrate how MCF10A-Ras spheroids show distinct invasive modes in different matrices. Representative confocal fluorescence maximum projection of a phalloidin-stained MCF10A-HRas spheroid without a BM layer 24 hours after embedding in a 3D collagen I matrix (1PT) at lower (FIG. 6A, scale bar=200 μm) and higher (FIG. 6B, scale bar=50 μm) magnification shows extensive individual invasion of cancer cells into the surroundings. Representative confocal fluorescence maximum projection of a phalloidin-stained MCF10A-HRas spheroid without a BM layer 24 hours after embedding in a 3D BME matrix (3BME) at lower (FIG. 6C, scale bar=200 μm) and higher (FIG. 6D, scale bar=50 μm) magnification shows spherical outgrowth from the spheroid body without any cells leaving the bulk spheroid. Representative confocal fluorescence maximum projection of a phalloidin-stained MCF10A-HRas spheroid without a BM layer 24 hours after embedding in a composite 3D collagen I/BME matrix (1PT3BME) at lower (FIG. 6E, scale bar=200 μm) and higher (FIG. 6F, scale bar=50 μm) magnification shows multicellular invasion with strongly polarized leader cells.

FIG. 7A-FIG. 7I show differential effects of MMP inhibition as a function of ECM. Representative confocal fluorescence maximum projections of solvent control (FIG. 7A) and MMP inhibitor-treated (FIG. 7B) phalloidin-stained MCF10A-HRas spheroids without a BM layer 24 hours after embedding in a 3D collagen I matrix. Scale bar=200 μm. FIG. 7C is a graph showing that quantitative analysis of MCF10A-HRas spheroid invasion in collagen I under MMP inhibition reveals no significant difference (p<0.05) in invasive distance between the treated and control groups. Invasive distances are shown with box plots depicting first to third quartiles with median denoted by a line and mean with a symbol; whiskers show minimum and maximum values, sample number n≥12 for every condition. Representative confocal fluorescence maximum projections of solvent control (FIG. 7D) and MMP inhibitor-treated (FIG. 7E) phalloidin-stained MCF10A-HRas spheroids with a BM layer 24 hours after embedding in a 3D collagen I matrix. Scale bar=200 μm. FIG. 7F shows that quantitative analysis of BM-shelled MCF10A-HRas spheroid invasion in collagen I under MMP inhibition reveals significant differences in number of collective invasion sites (lower panel) and number of individual cells (upper panel) invaded in the collagen matrix per spheroid with p<0.05 obtained in the non-parametrical Mann-Whitney test. Histograms show the percentage of spheroids with stated number of collective invasion sites or individual invaded cells in the inhibitor-treated versus the control group. n≥17 for every condition. Representative confocal fluorescence maximum projections of solvent control (FIG. 7G) and MMP inhibitor-treated (FIG. 7H) phalloidin-stained MCF10A-HRas spheroids without a BM layer 24 hours after embedding in a composite collagen I/BME matrix show abrogation of invasion under MMP inhibition. Scale bar=200 μm. FIG. 7I shows that quantitative analysis of MCF10A-HRas spheroid invasion in composite collagen I/BME matrix under MMP inhibition reveals significant difference in invasive area between the treated and control groups, with p<0.05 obtained in the Mann-Whitney test. Invasive areas for inhibitor and control groups are shown with box plots depicting first to third quartiles with median denoted by a line and mean with a symbol; whiskers show minimum and maximum values, sample number n≥6 for every condition.

FIG. 8A-FIG. 8B shows an example of assessment of invasive cross-sectional area from confocal fluorescence maximum projections of phalloidin-stained MCF10A-HRas spheroids without a BM shell 24 hours after embedding in collagen I (FIG. 8A) and BME (FIG. 8B) matrices. Individual invasion (FIG. 8A) was assessed by introducing a circle including ˜98% of the invaded cells as judged by visual inspection while collective invasion (FIG. 8B) was assessed by tracing the periphery of invasive structures and spheroid. In both cases, spheroid cross-sectional area measured at t=2 hours was subtracted from that measured at 24 hours to account for differences in initial spheroid size. Scale bars=200 μm.

FIG. 9 is a representative confocal reflectance image of a MB468 spheroid without a BM layer imaged 2 hours post implantation in a collagen I matrix showing radial alignment of collagen fibers towards the spheroid surface (red arrows) indicative of cell-mediated binding and force generation on the collagen matrix. Scale bar=50 μm.

FIG. 10A-FIG. 10D show representative (left) confocal fluorescence and (right) transmitted light images of early BM formation around MB468 spheroids performed without fluorescently labeled collagen IV (FIG. 10A) or with addition of 0.0025 mg/ml (FIG. 10B), 0.0050 mg/ml (FIG. 10C), or 0.0075 mg/ml (FIG. 10D) fluorescently labeled collagen IV. Images show spheroids in culture microplates 5 hours after initiation of the spheroid/BM formation process. White arrows indicate sites of incomplete BM compaction visible as irregular veil-like structures. Scale bar=200 μm.

FIG. 11A-FIG. 11C show representative confocal fluorescence maximum projection of a BM-shelled MCF10A spheroid 24 hours (FIG. 11A) and 48 hours (FIG. 11B) after embedding in collagen I gel with BM generated with low amounts of collagen IV (0.003 mg/ml), thus leading to a BM shell of limited thickness and density. Images show the structural integrity of the BM shell around the MCF10A spheroid after 48 hours of incubation under standard 3D culture conditions. FIG. 11C shows confocal fluorescence maximum projection of the BM-shelled MCF10A spheroid shown in FIG. 11A-FIG. 11B at 48 hours. Spheroid architecture was visualized by staining the actin cytoskeleton using phalloidin-AlexaFluor dye. The image shows the integrity of the spheroid and the absence of invasive structures 48 hours post-implantation in a collagen I gel. Scale bar=200 μm.

FIG. 12A-FIG. 12C show representative confocal fluorescence maximum projections of phalloidin-stained MB468 spheroids without a BM layer 24 hours after embedding in a 3D collagen I matrix (1PT) (FIG. 12A), a BME matrix (3BME) (FIG. 12B), or a composite collagen I/BME matrix (1PT3BME) (FIG. 12C). Images show extensive individual invasion in collagen I, no invasion in BME, and multicellular invasive structures in collagen I/BME composite matrix. Scale bar=200 μm.

FIG. 13 shows a representative confocal fluorescence maximum projection of a phalloidin-stained BM-shelled MB468 spheroid 24 hours after embedding in a 3D collagen I matrix showing multicellular invasive structures (white arrows). Scale bar=200 μm.

FIG. 14A-FIG. 14D show the results of cells from two different patient samples plated on substrates to demonstrate differences between those from the cancer cell and non-cancer cell fractions. These cells show a clear difference in morphology between the organoid (FIG. 14A and FIG. 14C) and single cell (FIG. 14B and FIG. 14D) fractions. The organoid fractions display epithelial morphology while the single cell fraction is fibroblast like.

FIG. 15A-FIG. 15B show bare organoids with no basement membrane shell prepared from cancer and non-cancerous cell fractions. FIG. 15A shows the results of organoids formed from a non-cancerous single cell fraction at 1 hour (left panel) and 24 hours (right panel). FIG. 15B shows the results of organoids formed from a cancerous cell fraction at 1 hour (left panel) and 24 hours (right panel).

FIG. 16 shows matrix reorganization in invading spheroids as displayed by collagen fibril alignment in confocal reflectance images (left panel, 60× magnification) and cells invading from the spheroid in transmittance imaging (right panel, 60× magnification).

FIG. 17A-FIG. 17D show that collagen IV shells surrounding organoids are formed from non-cancerous cell fractions at collagen IV concentrations of 0.003 mg/mL (FIG. 17A) and 0.0075 mg/mL (FIG. 17B) and from cancerous cell fractions at collagen IV concentrations of 0.003 mg/mL (FIG. 17C) and 0.0075 mg/mL (FIG. 17D).

FIG. 18A-FIG. 18D show heterogeneity in shell size and shape existed across different samples. The images show organoids formed from non-cancerous cell fractions at collagen IV concentrations of 0.003 mg/mL (FIG. 18A) and 0.0075 mg/mL (FIG. 18B) and from cancerous cell fractions at collagen IV concentrations of 0.003 mg/mL (FIG. 18C) and 0.0075 mg/mL (FIG. 18D).

FIG. 19A-FIG. 19B show invasion of the shelled organoids prepared from cancerous cell fractions. The left panels show 60× confocal reflectance images illustrating collagen fibril alignment resulting from cell traction generation, typical of aggressive breast tumors, and the right panels show transmittance images of multicellular streams emerging from the shell. The spheroid edge is in the bottom right of each image.

### DETAILED DESCRIPTION OF THE INVENTION

Aspects of the experimental system described herein relate to a multicellular aggregate (e.g., spheroid or organoid) comprising one or multiple cell types—such as cancer cells or epithelial cells—that is at least partially and even fully surrounded with a cell-bound layer of basement membrane, assembled and cross-linked in a cell-mediated reaction from exogenously added components, and then embedded in a tunable 3D biopolymer matrix that can be supplemented with dispersed cells of another type, such as cells of the immune system. In some aspects, the system is a high throughput system.

In one embodiment, cells (one or various types, such as if mixed cell aggregates are intended) are brought into suspension and diluted to the concentration of 1*10³-1*10⁵ cells/ml medium (depending on the intended size of cell aggregate) in cold growth medium supplemented with basement membrane extract (BME) at 0.2-0.4 mg/ml and collagen IV (fluorescently labelled if visualization of the basement membrane is intended) at 0.001-0.01 mg/ml. In one aspect, the formation of basement membrane-surrounded cell aggregates can be initiated by centrifuging the cell suspension in U- or V-bottom shaped, ultra-low adhesion multi-well plates, which may be coated with 2-methacryloyloxyethyl phosphorylcholine (MPC) polymer to prevent cell adhesion and protein deposition at the well surface, at 4° C. and 900-1300×g, until efficient sedimentation of cells and matrix components is achieved (the duration of centrifugation may depend on the format of the multi-well plate and thus the used volume). In another embodiment, cells are brought into suspension and diluted to a concentration of 2*104 cells/mL in cold growth medium. This suspension is plated at 100 μL/well in a U-bottom, ultra-low adhesion multi-well plate and then centrifuged at 1000×g for 10 minutes at 4oC. Alternatively, plates can be coated with poly(2-hydroxyethyl methacrylate) (Poly-HEMA) to prevent protein adsorption. Plates are then carefully removed from the centrifuge and 100 μL/well of cold media supplemented with basement membrane extract and fluorescently labeled collagen IV is added such that the final concentration of collagen IV in each well is between 0.001-0.015 mg/mL depending on the cell line, and the final concentration of basement membrane extract is set such that the final protein concentration in each well is between 0.2-0.3 mg/mL. The plates are again centrifuged at 4oC at 1000×g for 10 minutes. According to one aspect, multicellular aggregates of 100-500 μm in diameter with a basement membrane layer are fully formed after 18-24 hours incubation at cell culture conditions (37° C. and appropriate CO₂), and can be embedded into a 3D extracellular matrix (ECM) for further culture, treatment and monitoring. According to certain aspects, cell treatment with any agents can occur before aggregate formation (for example genetic manipulations) or after the aggregate is formed but before embedding in the 3D matrix as well as within the 3D matrix either immediately at time of embedding or at a later time point. In some aspects, the basement membrane is assembled by the cells.

In one embodiment, the matrix can consist of collagen I (0.5-4 mg/ml), BME (3-10 mg/ml), a mixture of collagen I and BME in various proportions, or any other polymer matrix that can be gelled at 37° C. or lower, and does not necessitate addition of cytotoxic agents or UV for gelation. The 3D matrix can contain an additional cell type (such as fibroblasts, immune cells etc.), added prior to or after the gelation and can be supplemented with agents targeting either cell function (chemotherapeutics, cytokines, antibodies and small molecule compounds etc.) or ECM (matrix degrading or crosslinking enzymes, soluble or modified matrix components etc.). In one embodiment, the 3-D extracellular matrix is a biopolymer comprising collagen I, collagen IV, basement membrane extract (BME), or a combination thereof.

In one embodiment, this experimental system can be used to screen or test existing drug candidates targeting both in situ and metastasizing solid cancers, specifically viability and migratory capacities of the cells in physiologically relevant 3-D environments. According to another embodiment, it can be utilized to test the capability of therapeutic agents to penetrate the tumor-associated basement membrane and exert their function on the solid or invading tumor. In yet another embodiment, the impact of the cell-polymerized basement membrane and its degradation products on cell-based therapeutic approaches (immuno-therapy) can be addressed using this model. Moreover, according to one embodiment, this system can be used with patient-derived tumor and/or immune cells as a platform for predicting tumor aggressive potential and patient-specific therapeutic responses.

According to yet another embodiment, the system may be used with multiple different cell lines of various different types, and may be used with patient-derived human tumor samples.

According to one embodiment, aspects of the invention relate to a newly developed physiologically relevant in vitro model of tumor progression to advance understanding of cellular metastatic mechanisms and to develop novel powerful and efficient platforms to test new treatment strategies and assess patient-specific responses to particular therapies. A vast majority of promising drugs fail in clinical trials: this highlights the fact that standard screening methods do not accurately predict human drug response (Hay et al 2014). Despite known poor predictive potential, 2D cell culture remains the standard for early stage drug screening, leading to resource intensive study of therapies destined to fail clinical trials (Breslin et al 2013). To speed efficient identification and development of both general chemotherapeutics and those tuned to particular patients, there is a need for new platforms to bridge the gap between the rudimentary, poorly predictive 2D systems and the physiologically relevant but time-intensive animal studies. This may be particularly crucial for development of immuno-therapeutic approaches since the behavior of immune and cancer cells is modulated by the properties of the extracellular matrix (ECM) and the specific oxygen- and cytokine-gradients characteristic in 3D tumor architecture.

Embodiments of the system described herein provide a physiologically relevant, easily manufacturable, and time-efficient in vitro model for tumors of epithelial origin at different stages of tumor progression. Aspects may be used for testing new pharmacologically and immune cell-mediated treatments against breast, colon and other solid tumors. Aspects also have the potential to be used with patient-derived tumor material in order to predict patient-specific treatment responses on a timescale of days, in contrast to animal-based tests that require several months.

In one embodiment, cell biological and tissue engineering approaches are combined to develop an experimentally accessible in vitro system for identification of effective therapeutic candidates at early stages as well as for assessment of personalized treatment responses in cancer patients. In one embodiment, the model offers a physiologically relevant 3D setting that combines i) a tumor mass architecture reflecting solid tumors in its cell-cell and cell-ECM contacts as well as in the oxygen- and cytokine gradients (Kimlin et al 2013), ii) a cell-bound basement membrane (BM) surrounding the tumor mass characteristic for cancers of epithelial origin (Kelley et al 2014) and iii) a hydrogel-based 3D ECM (Guzman et al 2014). The hydrogel allows for straightforward addition and monitoring of other cell types such as macrophages or T-cells to determine the effect of these cells on cancer cell behaviors such as invasive capacity (Linde et al 2012).

The tumor microenvironment including the tumor-bound BM and the surrounding ECM are increasingly being functionally linked to cancer-associated immune responses and to tumor progression and clinical outcome (Pickup et al 2014). In past work, tumor spheroids have been embedded in homogeneous hydrogels, recapitulating the ECM but not recapitulating the tightly bound BM and its degradation products as exist in solid tumors. Accordingly, in one embodiment the model described herein provides a tumor spheroid surrounded with a cell-bound BM assembled and cross-linked in a cell-catalyzed reaction and then embedded in a tunable 3D biopolymer matrix, potentially containing cells of the immune system. Aspects of this model of the tumor and its microenvironment do not require complex microfluidic approaches, and may be suitable for parallel processing (96-wells and higher). Aspects of this model may also provide a multicomponent system that combines high physiological relevance with a straightforward workflow that achieves low resource consumption and requires only a short time for experiment execution.

An embodiment of a system according to aspects of the invention using a “cell in shell in gel” model has been developed for use in a variety of breast cancer and healthy breast epithelial cell lines. The cell in shell in gel model may allow for the crafting of “mini-tumors” that are surrounded by a thin layer of basement membrane in advance of embedding in a second biopolymer gel matrix, which can be chosen to best mimic the extracellular environment of a particular tumor. The cell in shell in gel model may be flexible and straightforward to prepare, and may recapitulate not only the extracellular environment of solid tumors but also the hypoxic environment in which they develop and proliferate.

In one embodiment, a method has been developed to surround multicellular tumor spheroids with a BM layer consisting of exogenously added BM components that are cell-assembled by the spheroids. These “mini-tumors” are then embedded into tunable 3D collagen matrices for extended culture and monitoring. Using this biochemically well-defined and optically accessible model allows for i) analyzing the effects of several drug candidates targeting solid and metastatic breast carcinomas as one of the most common solid cancers, and ii) establishing a co-culture system with human monocyte-derived macrophages and T-cells from peripheral blood that can be used as an immunotherapeutic testing platform. For i), in one embodiment various commercially available reagents with proven clinical efficacy against solid tumors can be applied and drug response can be validated through assessment of cell survival and proliferation, metastatic progression as reflected by BM degradation and cancer cell transmigration of the BM, and invasion into the adjacent matrix. To achieve ii), in one embodiment the conditions are established for co-culture and monitoring of immune cells within the hydrogel as part of the described 3D model. In one embodiment, validation of the successful co-culture can be performed by assessing BM breaching and tumor cell invasion in the presence of macrophages, which have been reported to exert pro-metastatic effects (Condeelis et al 2006, Cardoso et al 2014).

The recognition of the immense diversity in treatment response and thus prognosis between cancer patients with the same cancer type has led to increased efforts to develop personalized therapy strategies. Accordingly, in one embodiment the protocol developed for cancer cell lines can be optimized for use with patient derived tumor samples and generate BM/ECM-embedded primary organoids. Metastatic potential can be evaluated as reflected by organoid growth, events of BM breaching and primary tumor cell invasion into the surrounding matrix. In another embodiment, patient-specific therapy responses as reflected by cell viability and invasive behavior can be assessed as a function of specific treatments chosen based on tumor genetics as assessed through microarray analysis. In yet another embodiment, after successfully establishing use of patient samples in this model and acquiring sufficient data, the predictive power of this model is established by correlating organoid behavior +/−treatment with patient prognosis based on standard clinical protocols. Aspects may also include incorporating therapeutically modified immune cells into the hydrogel surrounding BM/ECM-embedded primary organoids, as a tool to test patient-specific anti-tumor activity of immuno-therapeutics.

In another embodiment, the invention provides a method for diagnosing the presence of tumorigenic cells in a subject comprising (a) obtaining cells from the subject; (b) incubating the cells under conditions sufficient to form a multicellular aggregate surrounded by a layer of basement membrane; (c) disposing the multicellular aggregate surrounded by the layer of basement membrane in a 3-D extracellular matrix; (d) culturing the 3-D extracellular matrix under conditions sufficient to support growth of the cells; and identifying the cells as tumorigenic cells if the multicellular aggregate breaches the layer of basement membrane into the 3-D extracellular matrix. In some aspects of this embodiment the cells obtained from the subject are primary tumor cells. In other aspects of this embodiment the tumorigenic cells are carcinoma cells. In other aspects of this embodiment the tumorigenic cells are breast cancer cells.

As used herein, a “subject” is a mammal, preferably, a human. In addition to humans, categories of mammals within the scope of the present invention include, for example, primates, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

In the present invention, reference to “cells” is context dependent and may include primary cells, including cells obtained by tissue biopsy and other known methods of obtaining cells from a subject and cells from cell lines and other similar sources of cultured cells.

In another embodiment, the invention provides a method of treating or ameliorating the effects of a cancer in a subject comprising diagnosing the presence of tumorigenic cells in the subject by any of the methods of the present invention and administering to the subject an effective amount of an anti-cancer drug. In some aspects of this embodiment, the anti-cancer drug is determined according to the methods of the present invention. In some aspects of this embodiment, the cancer is a carcinoma. In other aspects of this embodiment, the cancer is breast cancer.

As used herein, the terms “treat,” “treating,” “treatment” and grammatical variations thereof mean subjecting an individual subject (e.g., a human patient) to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

In the present invention, an “effective amount” or a “therapeutically effective amount” of a compound or composition disclosed herein is an amount of such compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a compound or composition according to the invention will be that amount of the composition which is the lowest dose effective to produce the desired effect. The effective dose of a compound or composition of the present invention may be administered as two, three, four, five, six or more sub-doses, administered separately at appropriate intervals throughout the day.

As used herein an “anti-cancer drug” is any agent with a therapeutic effect against a cancer. Such agents are well known to the person of ordinary skill in the art. For example, an “anti-cancer drug” may be a selected from the group consisting of an antibody or fragment thereof, a chemotherapeutic agent, an immunotherapeutic agent, a radionuclide, a photoactive therapeutic agent, a radiosensitizing agent, and combinations thereof. Such anti-cancer drugs can be administered to the subject, either simultaneously or at different times, as deemed most appropriate.

The invention being thus described, it will be obvious that the same may be varied in many ways. Such variations are not to be regarded as a departure from the spirit and scope of the invention and all such modifications are intended to be included within the scope of the following claims.

### EXAMPLES

### Example 1

### Materials and Methods

### Cell Lines and Reagents

MDA-MB468 (referred to as MB468) breast cancer cells were obtained from the American Type Culture Collection (Manassas, Va.). MCF10A and MCF10A-HRas cells were a gift from Professor Carol Prives (Columbia University, N.Y.). All cell culture reagents unless otherwise stated were obtained from Gibco (Grand Island, N.Y.). Ultra-low attachment plates were obtained from NOF American Corporation (Lipidure microplates) (Irvine, Calif.) or from Thermo Fisher Scientific (Nunclon Sphera microplates, pre-treated with 2% bovine serum albumin (BSA) to block protein absorption to plate surface) (Waltham, Mass.). Pepsin-treated (PT) bovine collagen I was obtained from Advanced BioMatrix (San Diego, Calif.) as a 5.9-6.1 mg/ml solution. Growth factor-reduced, phenol red-free basement matrix extract (BME)/Matrigel was obtained as an 8.9-10 mg/ml solution from BD Biosciences (San Jose, Calif.). Fluorescein-conjugated DQ type IV collagen was obtained from Life Technologies (Carlsbad, Calif.), dissolved in distilled, deionized H₂O (ddH₂O) and used as a 1 mg/mL solution. Other sources of fluorescently labeled type IV collagen can also be used in this step. HiLite488-conjugated laminin was obtained from Cytoskeleton Inc. (Denver, Colo.), dissolved in ddH₂O and used as a 1 mg/mL solution. 10× DMEM solution, sterile NaOH (1 N) and sodium bicarbonate solution (7.5%) were purchased from Sigma Aldrich (St. Louis, Mo.). Gibco 4-(2-hydroxyethyl)-1-piperazineethanesulfonic acid (HEPES) buffer (1 M) was obtained from Invitrogen (Carlsbad, Calif.). Protease inhibitor cocktail (P1860) was obtained from Sigma-Aldrich. Triton-X and marimastat (BB-2516) were obtained from EMD Millipore Chemicals (Billerica, Mass.). 10% buffered formalin phosphate was obtained from Fisher Scientific (Pittsburgh, Pa.). AlexaFluor-conjugated phalloidin was obtained from Invitrogen Life Technologies (Grand Island, N.Y.). Fluorescent carboxy-modified microspheres (FluoSpheres 1 μm, λ_{ex/em}=535/575 nm, 2% solids) were obtained from Thermo Fisher Scientific.

### Cell Culture

MCF10A and MCF10A-HRas cells were cultured in 1× DMEM/F-12 medium supplemented with 5% (v/v) horse serum, 1% (v/v) 100× penicillin/streptomycin/amphotericin B solution (MP Biomedicals, Solon, Ohio), 0.5 μg/mL hydrocortisone (Sigma-Aldrich), 10 μg/mL insulin (Sigma-Aldrich), 0.1 μg/mL cholera toxin (Sigma-Aldrich) and 20 ng/mL EGF (Sigma-Aldrich) at 37° C. with 5% carbon dioxide. MB468 cells were cultured in 1× high glucose DMEM medium supplemented with 10% (v/v) fetal bovine serum, 1% (v/v) 100× penicillin/streptomycin/amphotericin B solution and 1% (v/v) 100× non-essential amino acids solution at 37° C. with 5% carbon dioxide. All cells were sub-cultured when 70-80% confluent.

### Generation of Multicellular Tumor Spheroids

Shell-free spheroids were formed using a centrifugation method described previously (Ivascu et al 2006). In brief, cells were brought into suspension in culture medium containing 0.2575 mg/ml BME and centrifuged at 4° C. for 10 minutes at 1000-1200 g in a Sorvall desktop centrifuge in ultra-low adhesion U-bottom culture plates. Culture plates were then transferred to an incubator for 24 hours at 37° C. with 5% carbon dioxide, allowing spheroid compaction. Spheroids were then treated with Cell Recovery Solution (Corning, Corning, N.Y.) for 45-75 minutes at 4° C. (time depending on cell type) prior to embedding in 3D matrices.

Prior to treatment with Cell Recovery Solution, spheroids prepared as described above had a layer of BME of variable thickness, density, and continuity, making them unsuitable for study of cell breaching BM. Thus, a variation of this method was developed to prepare fully shelled spheroids. To prepare spheroids fully surrounded by a BM layer, cells were brought into suspension in culture medium containing 0.2575 mg/ml total extracellular matrix proteins, consisting of 0.2500-0.2565 mg/ml BME and 0.0010-0.0075 mg/ml collagen type IV. Care was taken to ensure uniform distribution of BME and collagen IV in the solution, and perturbation of the solution after pipetting it into the culture plates was kept to a minimum. For formation of spheroids uniformly surrounded with a continuous BM, preventing adsorption of soluble matrix proteins onto the substrate was found to be critical. As such, Lipidure- or Nunclon Sphera-coated U-bottom 96-well plates additionally blocked with BSA were used. Centrifugation and transfer to the incubator was performed as described above for shell-free spheroids. Perturbation during transfer to the incubator was also kept to a minimum. Spheroid and shell were allowed to form for 24 hours under standard cell culture conditions. For preparation of fluorescently labeled spheroids, adherent cells were incubated with Vybrant DiD cell labeling solution (Thermo Fischer Scientific), diluted 1:200 in growth medium for 1 hour at 37° C., rinsed twice with PBS and processed as described in the spheroid preparation protocol above.

### Preparation of Hydrogel-Embedded Spheroids

Spheroids with or without a BM shell were prepared as described in the section above. Single spheroids were placed into one of three types of biopolymer solution (collagen I, BME, or composite collagen I/BME), each of which could then be gelled around the spheroid. Spheroids without a BM shell were placed in the solution directly after treatment with Cell Recovery Solution. Spheroids with a BM shell were washed with pre-warmed PBS 5 minutes at room temperature to remove loosely bound BM and debris before placement into the solution. Collagen I solutions at 1 mg/ml were prepared by diluting a high-concentration collagen stock solution. Appropriate amounts of collagen stock solution were prepared with 10% (v/v) 10× DMEM, 2.5% (v/v) HEPES buffer, 2.5% (v/v) sodium bicarbonate and ddH₂O. All solutions were held and mixed at 4° C. NaOH was added to adjust the pH to 7.4, and 200 μl of the neutralized collagen solution was immediately added to a chamber consisting of a 5 mm glass cylinder glued to a coverslip-bottom cell culture dish. A nylon mesh was placed on the inner circumference of the cylinder to anchor the gel. A single spheroid in 5 μl liquid was added to the liquid collagen. The gel chamber was then transferred to the 37° C. incubator. The collagen gels were overlaid with 50 μl growth medium after completion of gelation (t=1 hour) and surrounded by 700-1000 μl medium to prevent drying during extended monitoring following the incubation period. To prepare BME matrices loaded with a single spheroid, BME stock solution (8.9-10 mg/ml) was diluted with ice cold 1× DMEM to the final concentration of 3 mg/ml. 200 μl of the solution was added to a gel chamber and a single spheroid was added as described above. All steps were performed at 4° C. with pre-chilled solutions and instruments and transferred immediately to the 37° C. incubator. The gels were overlaid and surrounded with growth medium after 1 hour as described above. For composite collagen I/BME gels, first 10× DMEM, HEPES buffer, and sodium bicarbonate were mixed. Then, the required amount of BME stock solution was added to reach the final concentration of 3 mg/ml. The BME replaced a proportion of the ddH₂O that would be added in the equivalent pure collagen gel. Subsequently the collagen stock solution was added to achieve a concentration of 1 mg/ml, and the solution was brought to pH 7.4 by adding NaOH. After careful mixing, the solution was transferred to the chamber, a single spheroid was added and gelation and liquid overlay was performed as described above.

### Cell Treatments

Inhibition of endogenous proteases for cells cultured in 3D environments was achieved through addition of a protease inhibitor cocktail as described in Wolf et al 2003, using the P1860 inhibitor cocktail (Sigma-Aldrich) with additionally supplemented 100 μM marimastat. Spheroids were pre-treated with inhibitors or the respective solvent control diluted in growth medium for 2 hours at 37° C. in ultra-low adhesion plates before Cell Recovery Solution treatment (for spheroids without BM) and before washing steps (for spheroids with a BM layer). Both the collagen solution and the growth medium added on top of the 3D collagen matrix were supplemented with inhibitors at the same concentration as the pre-treatment solution.

### Microscopy

Spheroids and individual cells in 3D matrices were imaged with a 10× (NA=0.4) air and/or 60× (NA=1.42) oil objective on an inverted confocal laser-scanning microscope (Olympus Fluoview 300) in either scanning transmittance, confocal reflectance, or confocal fluorescence mode. An Argon ion laser at 488 nm was used for excitation of Fluorescein and HiLite488a and a Helium-Neon laser at 543 nm was used for excitation of AlexaFluor568. Fluorescence was detected on photo-multiplier tube detectors (PMT). Unlabeled collagen I was imaged via confocal reflectance microscopy (CRM) with the 60× oil objective using the 488 nm laser for excitation and a PMT for detection. Live cell imaging was performed using a custom-built microscope incubation chamber and objective heater to keep cells at 37° C. and 5% CO₂.

### Quantification of Imaging Data

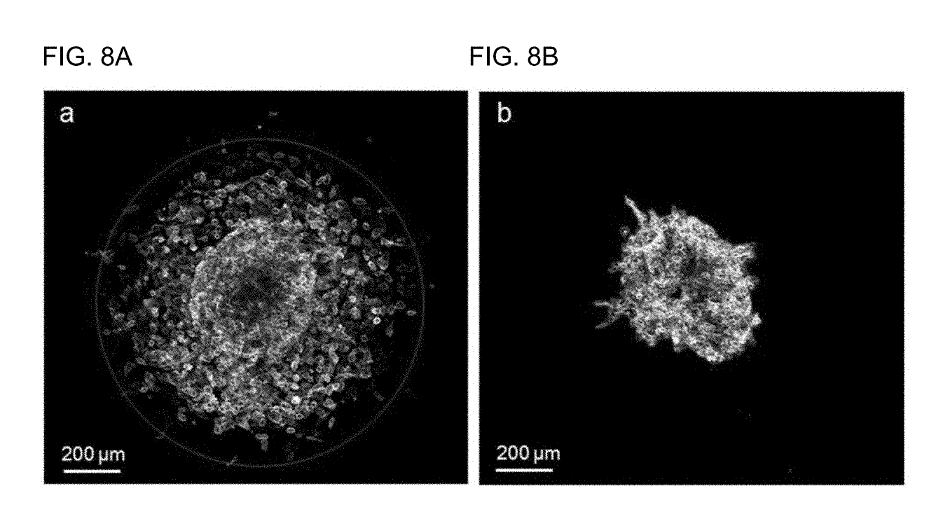

For quantitative assessment of invasion, spheroids were imaged in transmittance mode at 2 hours and 24 hours after implantation, with particular number of spheroids assessed noted in the figure captions. From the 10× magnification spheroid images, invasive distance for each spheroid was determined. Invasive area was defined as the difference between the area of the 2D projection of the spheroid at t=2 hours and t=24 hours. In cases with extensive individual cell invasion—as observed for spheroids without BM shells in pure collagen matrices—a circle was used to quantify invasive area (FIG. 8A). In cases with primarily collective invasion, as observed for spheroids without BM shells in composite collagen/BME matrices, invasive multicellular strand boundaries were traced, and the area of the resulting shape was used for further calculations (FIG. 8B). To assess the significance of differences observed between groups of 5<n<20, the non-parametrical Mann-Whitney-Wilcoxon test was applied as described in Fay et al 2010.

### Example 2

To investigate cellular migratory behavior during the initial steps of invasion under physiologically relevant and biochemically defined conditions, we developed and used a novel experimental model for multicellular cancer cell invasion that allows monitoring cancer cells breaching a cell-bound basement membrane and subsequently invading into a three-dimensional collagen-rich matrix.

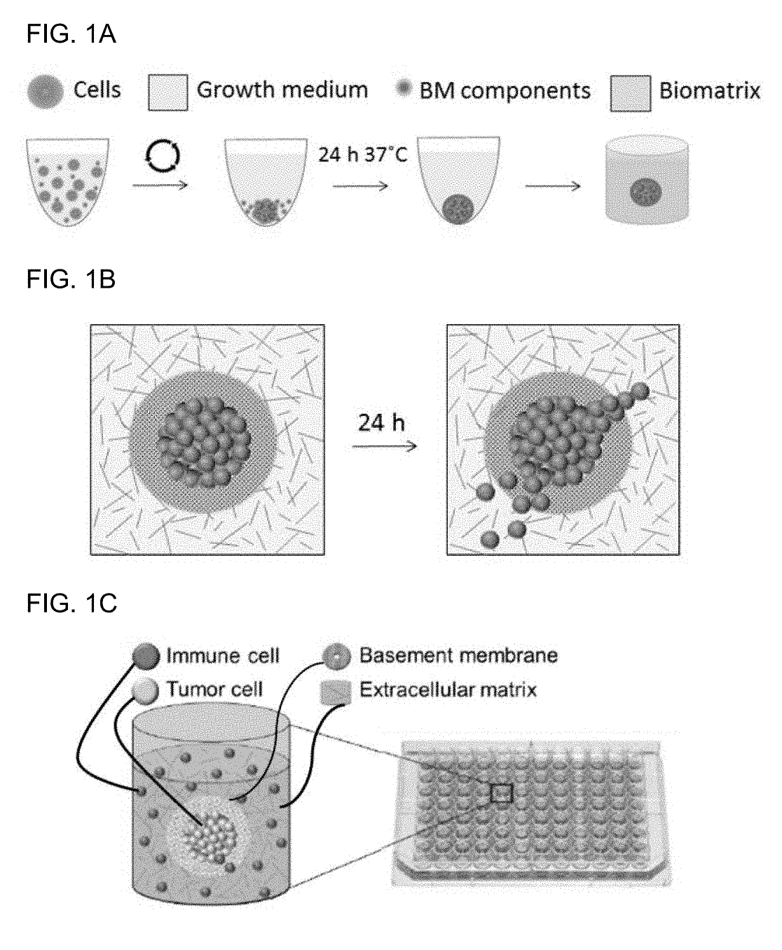

We have previously addressed breast cancer invasion using multicellular tumor spheroids embedded in 3D collagen, BME, or collagen/BME composite matrices (Guzman et al 2014, Ziperstein et al 2015). While these matrices represent appropriate models for cancer cell invasion in soft tissue, spheroid invasion into such environments does not recapitulate the serial nature of invasion in vivo, which requires breaching of BM before dissemination into stromal ECM. Thus, we developed a protocol for surrounding spheroids with a BM layer of tunable thickness and subsequently implanting those shelled spheroids into 3D biopolymer matrices in which BM transmigration and ECM invasion can be monitored (FIG. 1).

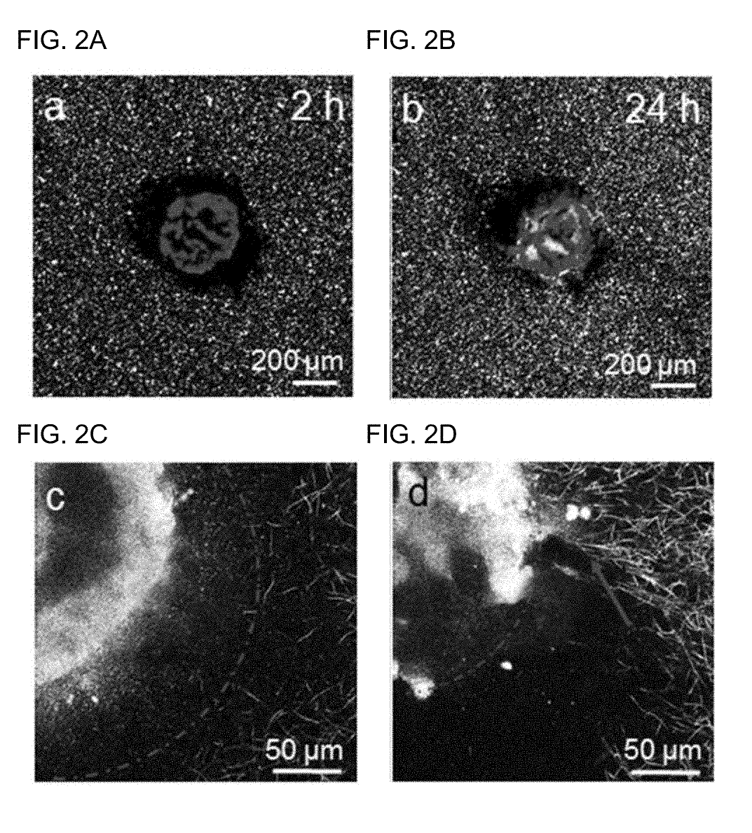

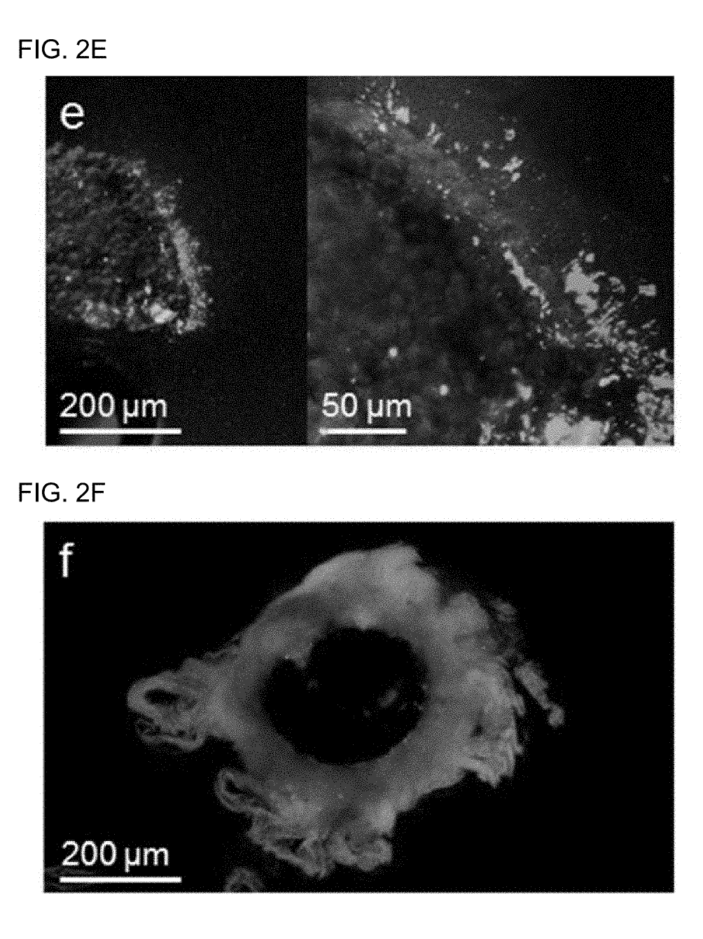

To assess the size of the BM layer and its integrity over time, BM-shelled spheroids were embedded into 1 mg/ml collagen I gels loaded with 1 μm fluorescent beads. These beads are smaller than the pores of the collagen I matrix but larger than those in the BM layer and are thus excluded from that layer. Confocal fluorescence microscopy (CFM) of an MB468 spheroid prepared with a BM shell showed a roughly circular area significantly larger than the spheroid from which beads were excluded (FIG. 2A). Imaging over the height of the spheroid indicated that the BM layer enveloped the spheroid. While the spheroid was shielded from the bead-loaded collagen I initially, sites of bead accumulation at and within the spheroid could be observed 24 hours after embedding, suggesting that the BM layer was partially degraded and the tumor cells were interacting with the surrounding, bead-loaded collagen I matrix (FIG. 2B).

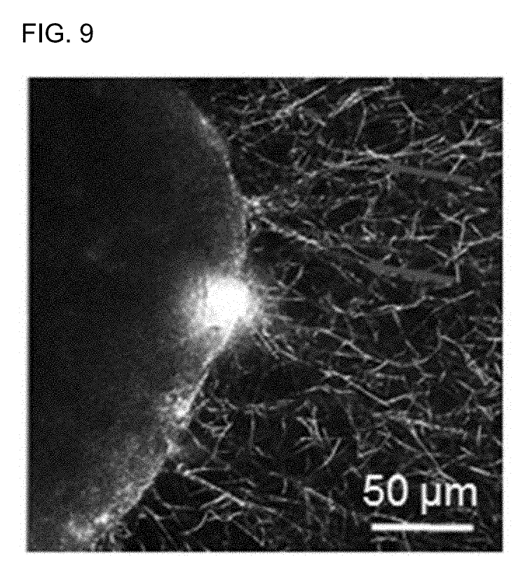

To more fully establish whether the BM shell prevents the spheroid from direct contact with the collagen I matrix, spheroids with a BM shell were implanted into collagen I matrices and subjected to confocal reflectance microscopy (CRM), which allows visualization of unlabeled collagen fibers but not of non-fibrillar substrates such as BM. Indeed, the spheroids with BM shells displayed an area beyond the spheroid periphery devoid of collagen fibers, and the collagen fibers closest to the spheroids were isotropically arranged (FIG. 2C). This was in contrast to spheroids without a shell, which had collagen fibers adjacent to the spheroid surface that were radially aligned, indicating traction generation by cells in the spheroid (FIG. 9). Once the BM shell was breached and cells had established direct contact with the collagen matrix, radially aligned collagen fibers at the spheroid surface of initially shelled spheroids were also apparent (FIG. 2D).

We next addressed whether formation of the BM layer resembles the process in vivo, relying on laminin for the initial scaffold and collagen IV as a central structural component (Kalluri 2003). To this end, fluorescently labeled laminin or type IV collagen was introduced into the medium during spheroid formation, reducing the amount of unlabeled BM proteins accordingly to keep the total concentration of exogenous BM components constant. Accumulation of fluorescent material around the spheroid was analyzed using CFM 2 hours after implanting the spheroids in the surrounding gel. Laminin could only be detected at the spheroid surface, displaying patches of varying size and not constituting a continuous layer (FIG. 2E). On the other hand, collagen type IV was present as a largely homogeneous layer of approximately uniform thickness around the spheroid (FIG. 2F). The BM layer was found to be resistant to dissolution with mild chaotropic agents that efficiently dissolve cell-independently formed BME gels that rely on non-covalent forces for structural integrity.

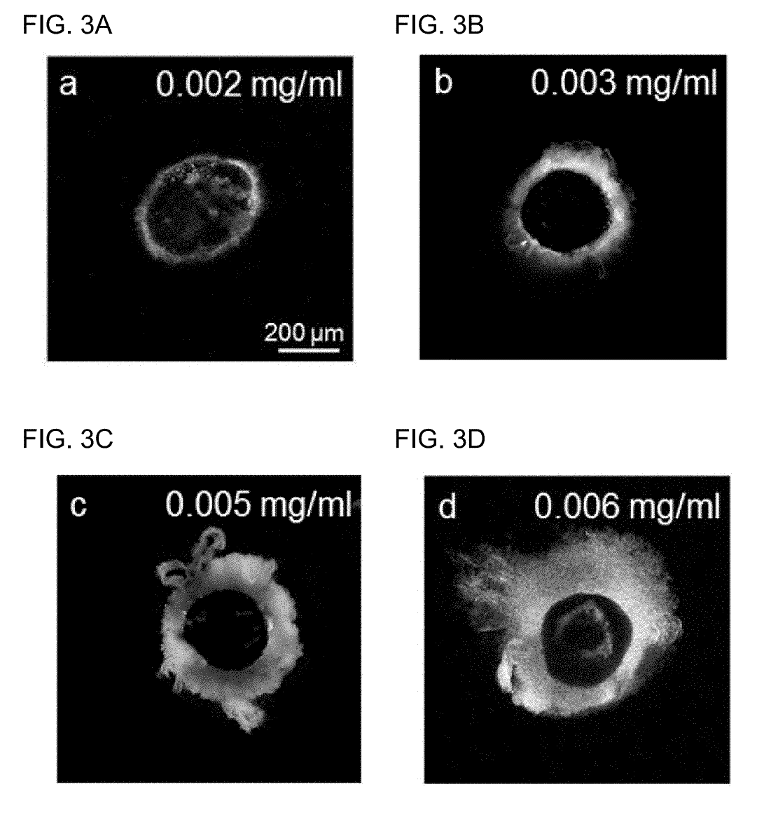

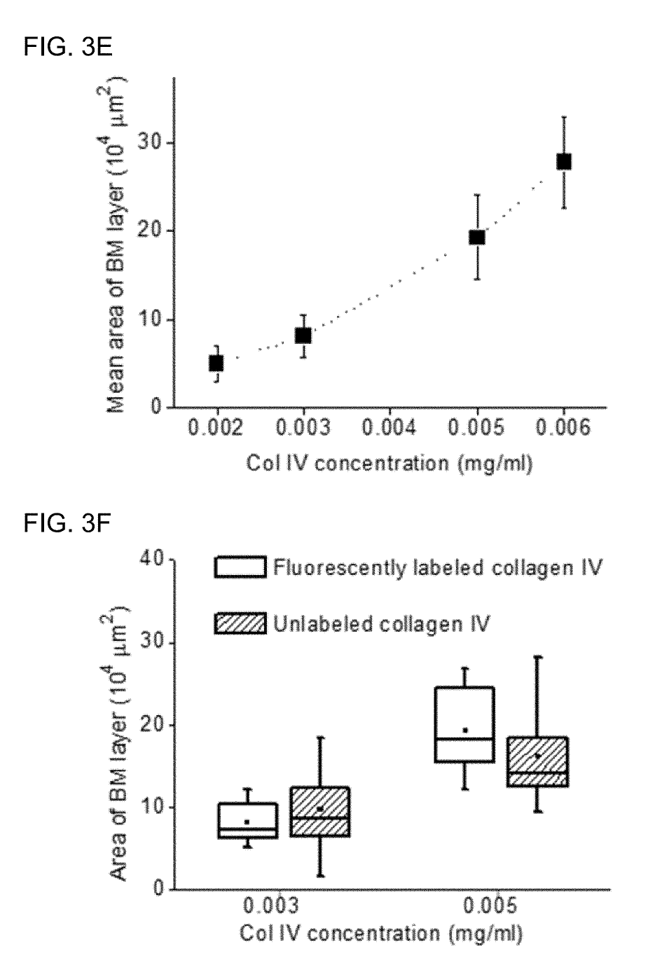

To test whether the formation of the BM shell relies mainly on exogenously added BM components or on endogenous production of these proteins, correlation between shell size and the concentration of supplemented type IV collagen was investigated. Spheroids were supplemented with varying amounts of fluorescently labeled type IV collagen for the duration of spheroid/BM layer formation (24 hours) and subsequently subjected to confocal fluorescence/transmittance imaging. This approach revealed that the BM thickness is strongly dependent on the concentration of exogenous type IV collagen (FIG. 3A-FIG. 3D). For quantitative assessment, the cross-sectional area of visualized collagen IV was assessed from a maximum fluorescence intensity projection over the full height of the spheroid and plotted versus concentration of exogenously added collagen IV. This analysis shows a direct correlation between this quantity and the concentration of supplemented collagen IV (FIG. 3E). Moreover, shell size as defined by the area in which fluorescently labeled collagen IV is present is comparable with the collagen I-devoid areas observed at the same BM-formation conditions in bead-exclusion experiments as shown in FIG. 2A (FIG. 3F), demonstrating that collagen IV is the main structural component of the BM shell.

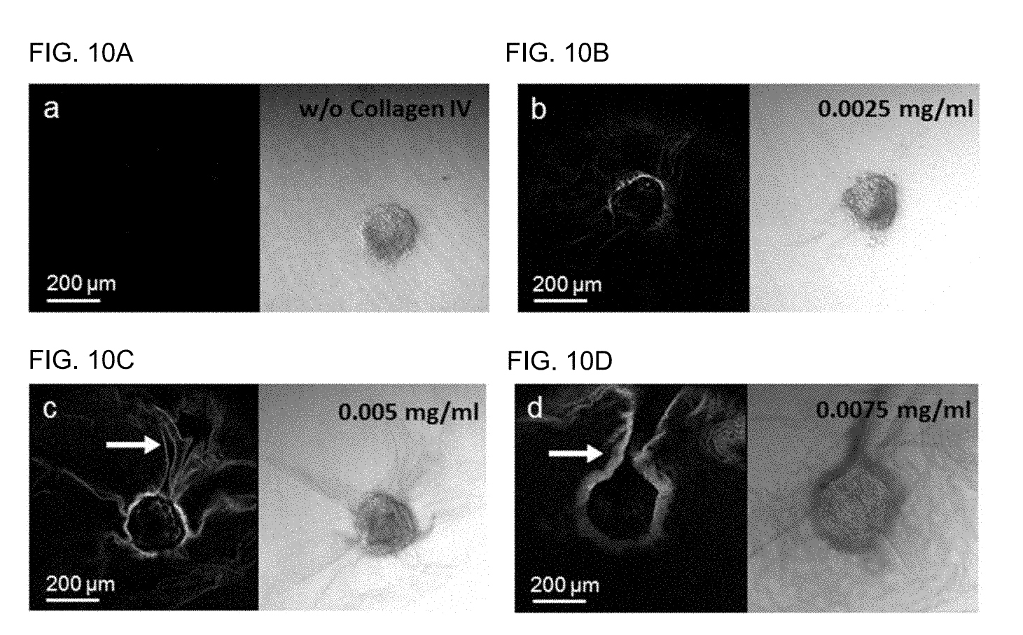

Time lapse imaging revealed the time course of the shell formation process. Fluorescently labeled collagen IV accumulated around the spheroid as early as 4-5 hours after process initiation. At such early time points, collagen IV was present in irregularly shaped veil-like structures emanating from the spheroid surface (FIG. 10A-FIG. 10D). These structures were compacted into a denser, more uniform layer within 24 hours at collagen IV concentrations 0.003-0.006 mg/ml (FIG. 3). We note that at concentrations higher than 0.006 mg/ml collagen IV, complete compaction did not always occur and irregular shell extensions sometimes remained after 24 hours (data not shown). Observation of the BM structures at these early time points reveals a similar correlation between basement membrane density and size and collagen IV concentration as observed at the later time point as shown in FIG. 3. Taken together, these results indicate that the BM surrounding spheroids is primarily formed from the exogenously supplemented BM components.

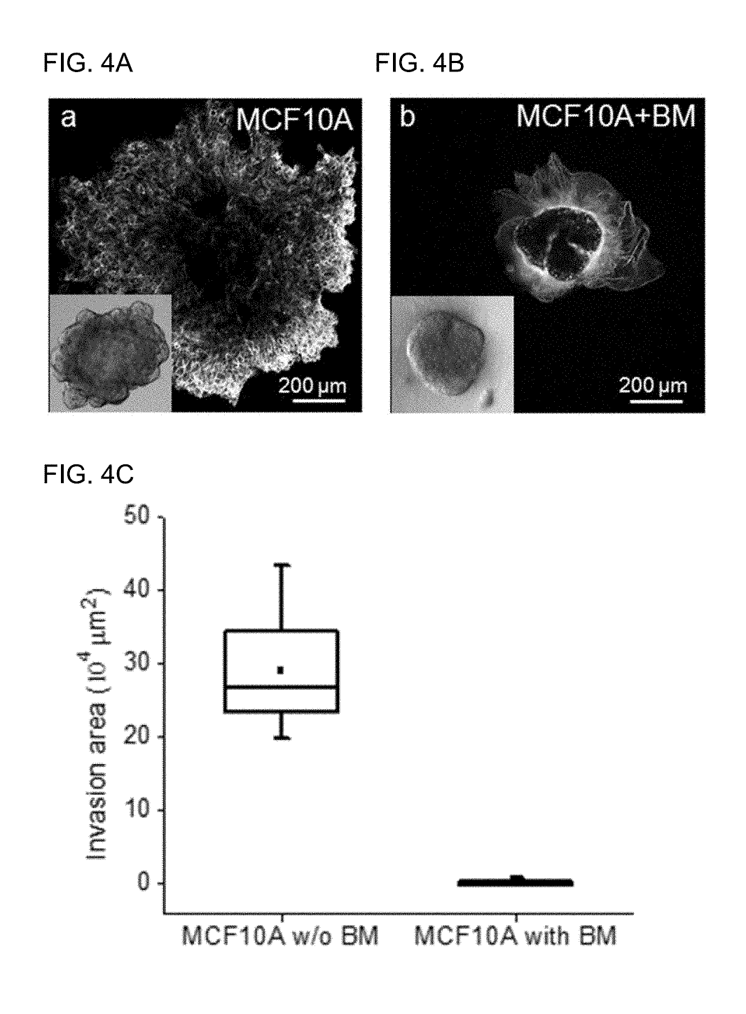

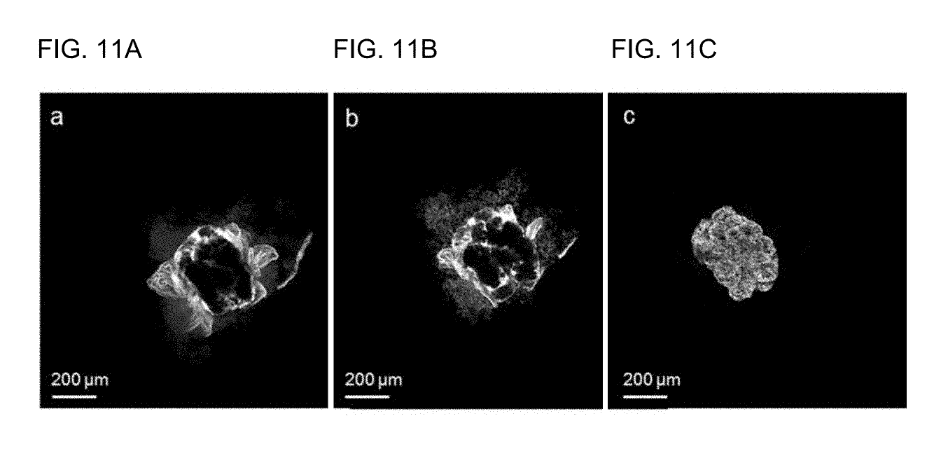

We next interrogated whether the BM layer around the spheroid mimics the function and behavior of BM in vivo, where this layer separates healthy cells from the surrounding tissue containing them within its boundary but can be degraded and traversed by cancerous cells. To this end, spheroids with BM shells were generated from non-tumorigenic and from oncogenically transformed breast epithelial cells with the same genetic background, namely MCF10A and MCF10A-HRas cells. These spheroids were embedded into 3D collagen I matrices and monitored for the integrity of the BM layer as well as cell invasion into the collagen matrices up to 48 hours after embedding. BM shell structure was visualized via confocal fluorescence microscopy of labeled type IV collagen, while spheroid architecture and cell dissemination were visualized either via transmitted light imaging or CFM following immunofluorescent staining of actin cytoskeleton. As a control, spheroids without a BM layer were used. Collagen-embedded non-cancerous MCF10A spheroids with no BM layer exhibited sheet-like expansion with a closed cell front and no individual cell invasion into the collagen (FIG. 4A). In contrast, spheroids with BM shells remained fully confined within the boundary defined by the BM throughout the monitoring period and exhibited negligible increase in spheroid cross-sectional area over 24 hours (FIG. 4B); complete abrogation of cell dissemination into collagen I was effected through the introduction of the BM layer (FIG. 4C). This behavior was also observed for spheroids with a BM layer formed at the lowest collagen IV concentration yielding a complete shell (0.003 mg/ml), indicating that the presence of a BM layer does not merely reduce but abolishes the migratory activity of MCF10A cells (FIG. 11A-FIG. 11C).

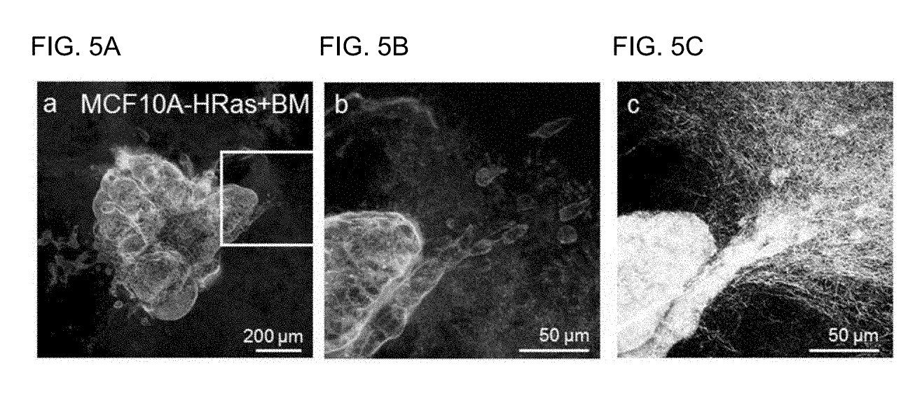

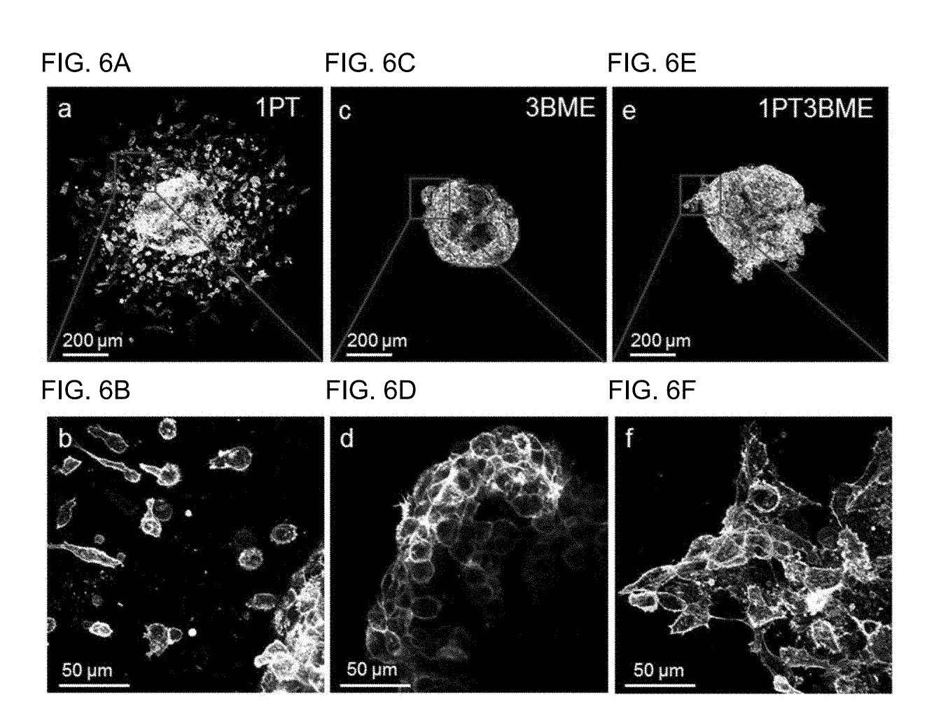

In contrast to MCF10A spheroids, the oncogenically transformed MCF10A-HRas spheroids were not contained by the presence of the BM layer and exhibited multiple BM breaching events and dissemination of cells into the collagen matrix within 24 hours after embedding (FIG. 5A). Compared to MCF10A-HRas spheroids with no BM layer embedded in collagen I, the density of invading cells was greatly diminished (FIG. 5A and FIG. 6A). This is consistent with the hypothesis that the presence of a BM layer presents a biophysical challenge for cells, which must first breach this layer before invading into the collagen I environment. Interestingly, cancer cells displayed a much higher incidence of multicellular invasion when transmigrating the BM layer, displaying streams of densely packed cells moving through the BM towards the collagen matrix, than during invasion in absence of a BM layer, where cells exclusively depicted individual mesenchymal invasion (FIG. 5B and FIG. 6A-FIG. 6B). The invasive streams, once in contact with the collagen I matrix, exhibited strong traction on the collagen matrix as reflected by the accumulation and high degree of alignment of collagen fibers at the tip of the invasive strand (FIG. 5C).

The combined use of collective and individual invasion modes by spheroids with a BM shell is fundamentally different from invasion observed for 3D-embedded spheroids in the absence of a BM layer. While collagen I matrices support strong individual invasion and mesenchymal cell morphology (FIG. 6A-FIG. 6B), a matrix composed solely of basement membrane extract (BME) does not support any invasive behavior over the assessed time scales (FIG. 6C-FIG. 6D). In composite collagen I/BME matrices, the cells show collective invasion as well as strong cell polarity similar to the initial migratory phenotype of shelled spheroids during invasion through the BM layer (FIG. 6E-FIG. 6F); however this matrix does not support the switch to individual invasion at any point.

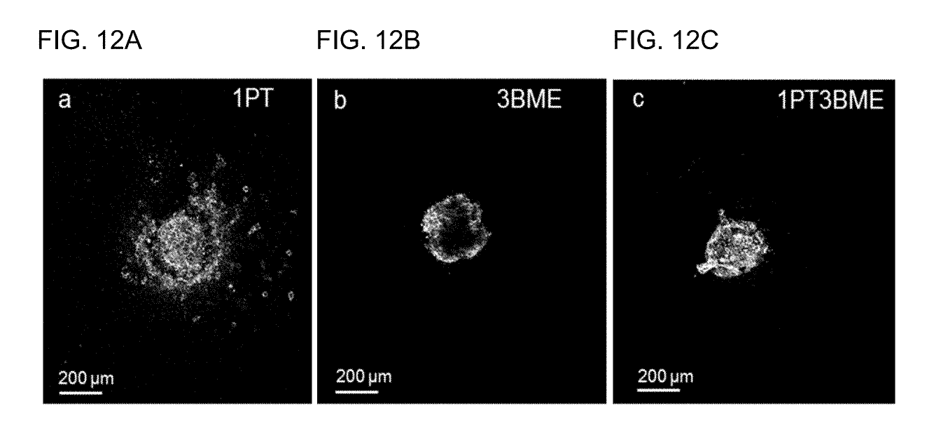

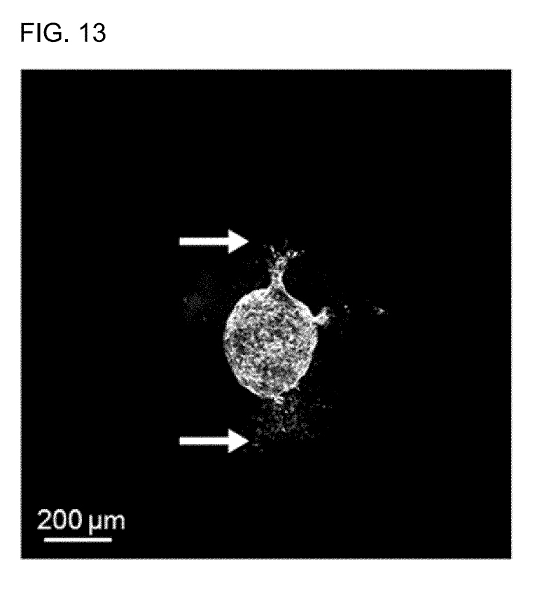

Next, we investigated whether the observed induction of collective invasion in shelled spheroids relative to unshelled spheroids in collagen I environments is a cell-type specific response to these experimental conditions or a more general behavior of tumorigenic cells in the presence of a BM layer. Thus, invasion studies similar to those performed on MCF10A-HRas were performed on cancer cells of different origin, namely MB468 breast cancer cells. MB468 spheroids prepared without a BM layer and introduced into collagen I matrices showed individual cell invasion into the surroundings, no invasion in pure BME matrices and multicellular invasion in composite collagen I/BME matrices (FIG. 12A-FIG. 12C), paralleling the invasion pattern observed in response to these matrices for MCF10A-HRas cells. When prepared with BM shells, the cells successfully traversed the BM layer and invaded into collagen (FIG. 13), as also found for MCF10A-HRas cells. While the two cancerous cell lines differ in origin, cell morphology (mesenchymal MCF10A-HRas and grape-like MB468 ) and time required for dissemination (24 hours for MCF10A-HRas, 24-48 hours for MB468 ), they were similar in their ability to breach and transmigrate the surrounding BM layer, in contrast to the non-tumorigenic MCF10A cells. Thus this new experimental model recapitulates the fundamentally different physiological behavior of cancerous and non-cancerous cell aggregates surrounded by a cell-bound BM.

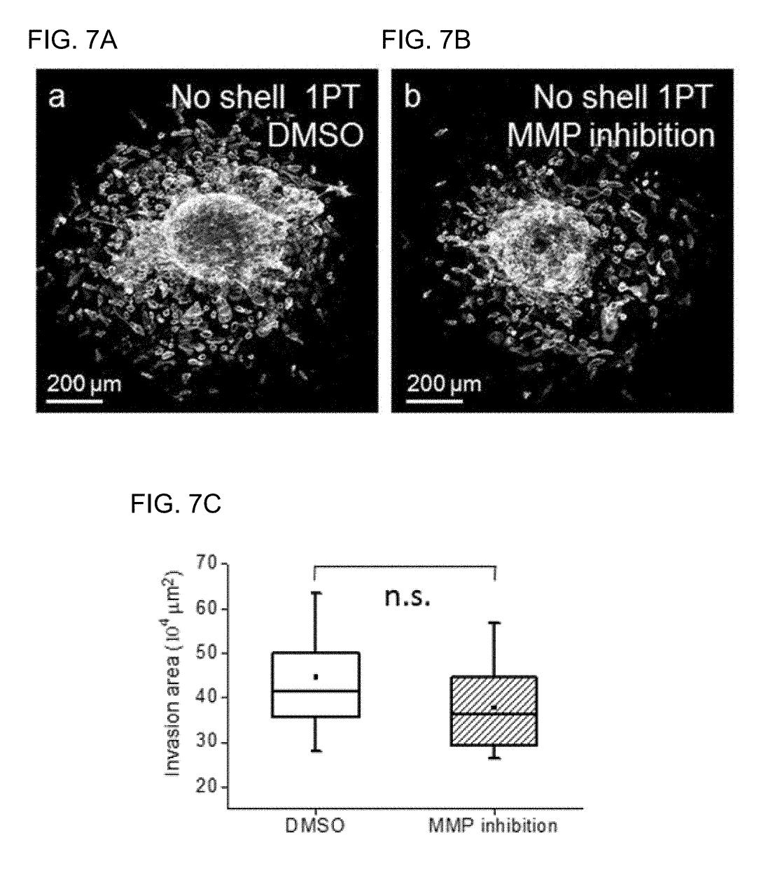

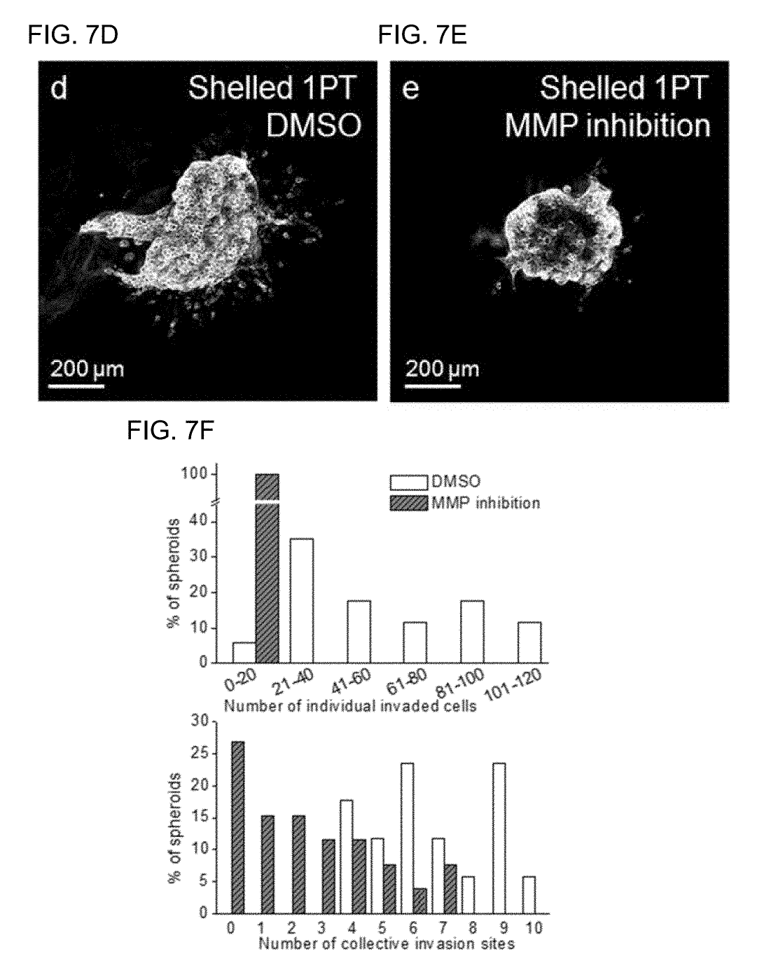

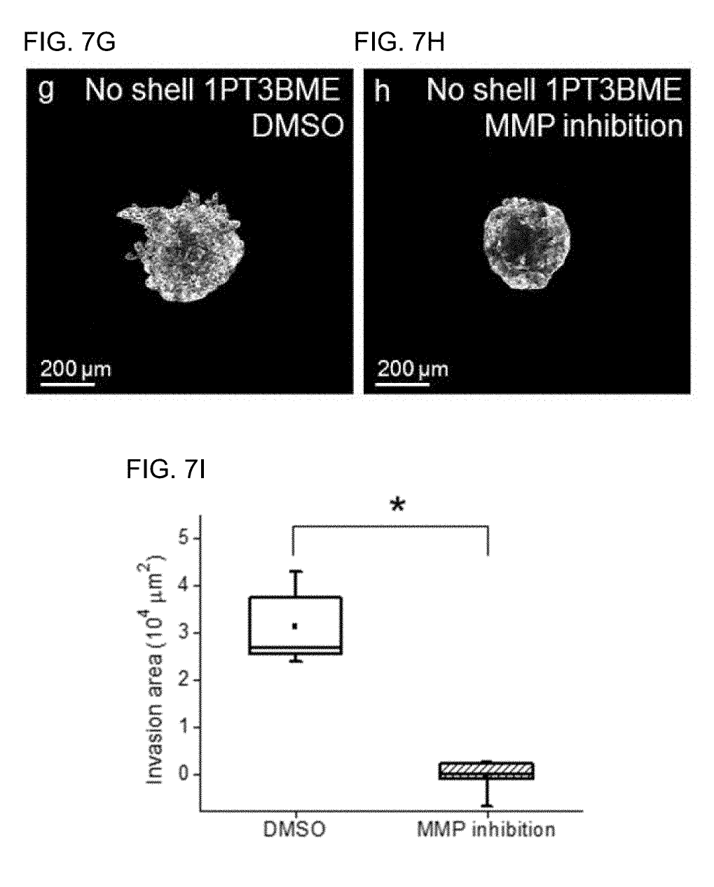

We next investigated molecular activity required for cells to traverse the BM layer, in particular whether matrix metalloproteinases (MMPs) were required for BM breaching and/or subsequent invasion in collagen I. To this end, MCF10A-HRas spheroids prepared with or without BM layers were pre-treated with an MMP-inhibitor cocktail targeting MMP-1, −2, −3, −7, −9 and −14 (MT1-MMP) as well as aminopeptidases and serine- and cysteine-proteases and embedded in collagen I matrices supplemented with the same inhibitors. We note that MCF10A-HRas has been reported to have upregulated expression of both MMP-2 and MMP-9, with the former regulated by MT1-MMP, relative to MCF10A. After 24 hours, samples were fixed and subjected to actin cytoskeleton staining and confocal fluorescence imaging as described earlier. It was found that the presence of the BM layer strongly modulated the cellular response to MMP inhibition. Collagen I invasion of spheroids without a BM layer was only mildly affected by MMP inhibition, with no observable differences of invasion mode or cell morphology and no significant reduction of invasive distance (FIG. 7A-FIG. 7C). In contrast, the invasive behavior of BM-shelled spheroids was strongly compromised by MMP inhibition. Here the MMP inhibition led to a greater than two-fold reduction of invasion incidence (from 100% to 38.5%), as characterized by the presence of individual cancer cells in the collagen matrix, and a nearly twenty-fold reduction in the mean number of individual invasive cells (57 to 2.5) (FIG. 7D-FIG. 7F). MMP inhibition in BM-shelled spheroids did not fully prevent formation of multicellular streams, while it did abolish formation of invasive structures (and invasion) in BM-free spheroids embedded in composite collagen I/BME matrices, the condition that induces multicellular invasion in the absence of MMP inhibition (FIG. 7G-FIG. 7I). While the multicellular structures in BM-enveloped spheroids still formed under MMP inhibition, this occurred in reduced numbers per spheroid (from median 6 to 2 per spheroid). Moreover, 50% of the multicellular streams failed to breach the BM layer within 24 hours, indicating that the efficiency of BM breaching is dependent on MMP-mediated BM degradation. The fact that multicellular invasive streams formed under MMP inhibition in BM-enveloped spheroids while their formation was completely abrogated in bare spheroids embedded in composite matrix suggests that ECM comprising a cell-bound non-fibrillar BM adjacent to fibrillar collagen matrix evokes a particular collective invasion mode that is more resistant to pharmacological MMP inhibition than spheroid invasion (without a BM shell) in a composite matrix consisting of a mixture of both components.

### Example 3

Despite decades of study, the cellular events that allow an in situ circumscribed tumor to become an invasive entity and the molecular mechanisms underlying the penetration of cancer cells through the BM and adjacent ECM are not fully understood. Here, we present an optically accessible 3D model that recapitulates diverse dynamic cell-cell and cell-ECM interactions that exist as cells traverse a dense, sheet-like BM layer in advance of invasion into adjacent ECM.

The experimental model presented in this study consists of spheroids containing several thousands of benign or tumorigenic cells surrounded by a BM layer and embedded into a biomechanically tunable 3D matrix (FIG. 1). Importantly, we found that non-tumorigenic cells were confined by the BM layer (FIG. 4B and FIG. 11A-FIG. 11C), while spheroids composed of various cancerous cell lines breached the BM layer and invaded into the adjacent matrix within 24 hours after embedding (FIG. 5A-FIG. 5C, FIG. 13). This recapitulates a critical process in the progression of metastatic disease. When a dysplastic carcinoma in situ acquires the ability to traverse the BM, the lesion is classified as a malignant carcinoma (Hanahan et al 2000, Rowe et al 2008, Bosman 1994). For probing the invasive behavior of cancer cells, the model of the present invention is superior to embedding spheroids in pure collagen I matrices, as some non-cancerous epithelial cells spread from the spheroid in 3D collagen I matrices despite their benign character (FIG. 4A). While the cells maintain tight cell-cell contacts and a closed cell front more reminiscent of sheet expansion than true invasion in a collagen matrix, the spheroid—in absence of a BM layer—loses its original architecture and does not fully reflect its non-tumorigenic nature. This is avoided by surrounding the MTS with a dense layer of BM (compare FIG. 4A and FIG. 4B).

Another critical advantage of the shelled spheroid system of the present invention is the structure of the BM layer, which is intimately related to the mechanism of its generation. Conventional assays probing cell invasion in BM commonly use basement membrane extract polymerized in a cell-independent manner. In contrast, in the shelled spheroids protocol of the present invention, the BM layer is assembled from supplemented components in a cell-mediated process. This process requires functional β1 integrin receptors since antibody-mediated β1 integrin inhibition strongly compromised the formation of a continuous and dense BM layer (data not shown). This is in accordance with the β1 integrin-dependent mechanism reported for BM formation in mice (Raghavan et al 2000) and suggests that the formation of the BM layer in the presented experimental system requires similar cellular mechanisms to the respective process in vivo. This hypothesis is supported by the observation that in the present model, laminin is bound and forms a thin patchy layer directly at the spheroid surface (FIG. 2E) while collagen IV is polymerized into a complex network that constitutes the bulk of the BM structure (FIG. 2F and FIG. 3). This closely recapitulates the reported molecular mechanisms of BM assembly in vivo, where laminin is polymerized at the cell surface and serves as the initial template for scaffold formation through type IV collagen polymerization (McKee et al 2007, Li et al 2005).

BME polymerized in a cell-independent manner is not only more compliant than endogenous BM (Soofi et al 2009, Halfter et al 2015), it also lacks some hallmarks of mature BM structure, such as covalently cross-linked collagen IV (Even-Ram et al 2005, Hotary et al 2006, Sodek et al 2008). Since matrix stiffness and architecture were shown to be of crucial importance for cancer cell invasion mode and efficiency in various studies (Guzman et al 2014, Wolf et al 2013, Zaman et al 2006, Petrie et al 2012), it is possible that cell invasive strategies observed in BME gels are not identical to those utilized by cancer cells traversing BM in vivo. This hypothesis is supported by our finding that cancer cells that are non-invasive in 3D BME gels can efficiently transmigrate the BM layer in the present model (compare FIG. 5A and FIG. 6C, FIG. 12B and FIG. 13). Furthermore, a study utilizing decellularized peritoneal BM demonstrated that transmigration of native BM requires a different subset of MMPs than invasion of in vitro reconstituted BM (BME gels) and—in contrast to BME invasion—cannot be abolished by inhibition of secreted MMPs (Notary et al 2006). Thus, it is particularly important to address the mechanisms of BM breaching in a system that not only recapitulates the biochemical composition of BM but also more closely resembles its biomechanical properties in vivo.

While many models suggest metastasis begins with individual cells undergoing the epithelial-mesenchymal transition (EMT) and leaving the boundaries of the primary tumor, analysis of tumor-stroma interfaces in clinical samples has revealed that it is the presence of invasive cell clusters (Hanahan et al 2011), also termed tumor buds, that correlates with metastatic progression and poor prognosis in various solid tumor types (Ohike et al 2010, Mitrovic et al 2012, Karamitopoulou et al 2013, Liang et al 2013, Sun et al 2014). This highlights the importance of understanding the cellular and molecular underpinnings of collective cancer cell invasion and the need for physiologically relevant in vitro models supporting this crucial mode of invasion. To date, in vitro settings for the study of collective cancer cell migration have relied primarily on 2D scratch/wound assays or on assays using spheroids or organoids embedded in 3D matrices, typically composed of collagen I or BME (Das et al 2015, Graves et al 2016, Kaufman et al 2005, Yang et al 2010, Nguyen-Ngoc et al 2012). We and others have reported differential invasive behavior for cancerous cells in fibrillar (collagen I) vs. non-fibrillar (BME) 3D matrices, with collagen I typically being more supportive of invasion than is non-fibrillar BME, which did not lead to invasion in either spheroids or organoids of known tumorigenic breast, ovarian and prostate cancer cells (Guzman et al 2014, Sodek et al 2008, Nguyen-Ngoc et al 2012, Harma et al 2010). Recently, we showed that one breast cancer cell line showed individual invasion in collagen I matrices, no invasion in BME, and a primarily collective mode of invasion in a composite collagen I/BME matrix (Guzman et al 2014). These results mirror those found in the MCF10A-HRas and MB468 cell lines shown here (FIG. 6A-FIG. 6F, FIG. 12A-FIG. 12C). Although the composite matrix appears to be a better system to evoke and study collective invasion than do homogeneous collagen I or BME matrices, a homogeneous environment does not recapitulate the in vivo setting in which cells are faced with distinct ECM components serially, as epithelial based tumors must first breach a cell-bound BM layer to subsequently migrate through stromal ECM.

Interestingly, we find that in MCF10A-HRas spheroids surrounded by a layer of cell-assembled BM, the formation of multicellular streams and a degree of successful invasion occurs under MMP inhibition targeting both secreted MMPs and the membrane-bound MT1-MMP. In contrast, this cellular behavior is completely abolished in unshelled spheroids embedded in composite matrices (FIG. 7). The lower sensitivity to MMP inhibition indicates that the cells utilize different invasion mechanisms during transmigration of cell-assembled BM than when confronted with BME polymerized in a cell-independent manner. This, together with the largely MMP-independent migratory mode of individual cancer cells in fibrillar collagen I matrix, offers a compelling explanation for the inefficiency of MMP inhibition as a treatment strategy for late stage cancers (Coussens et al 2002).

The model disclosed herein consisting of a spheroid with a discrete, cell-bound and assembled BM layer that may be embedded into a biomechanically tunable collagen matrix is believed to be a better approximation of the in vivo scenario than is any uniform hydrogel system. Supporting our hypothesis, the model disclosed herein supported multicellular streaming and collective invasion through the BM layer in tumorigenic cancer cell lines that did not show this behavior in pure collagen (FIG. 5A-FIG. 5C, FIG. 13). The enhanced collective behavior seen in the layered system vs. in a homogeneous BME matrix suggests that the BM layer polymerized by cell activity has matrix architecture, mechanical properties, and/or adhesion ligand presentation that supports migratory strategies that are less dependent on MMPs than typical strategies invoked in BME. The existence of a confining BM shell around the growing spheroid may also drive collective invasion by generating elevated pressure within the spheroid that mimics the high interstitial pressure observed in solid tumors, a characteristic that has been linked to altered migratory behavior in vitro and increased metastasis and poor prognosis in vivo (Tse et al 2012, Milosevic et al 2001, Hompland et al 2012, Polacheck et al 2014).

We have developed a novel experimental model in which tumor spheroids surrounded by a cell-bound BM of tunable thickness are generated and may be subsequently embedded in a second biopolymer matrix such that the cells serially encounter multiple, adjacent extracellular environments. Using this model, central initial events of metastatic progression were recapitulated in a physiologically relevant setting. First, we showed that tumorigenic breast cancer cell lines of two different subtypes can breach this BM within 24 hours, while non-cancerous breast epithelial cells were fully retained within BM borders, thus reproducing an early hallmark of metastatic behavior. We also demonstrated selective cancer cell utilization of collective migration for transmigration of the physically challenging BM layer. Moreover, this study revealed that while BM breaching, in contrast to collagen I invasion, is an MMP-dependent process, it is less susceptible to pharmacological MMP inhibition then collective invasion in homogeneous composite matrices and cannot be fully abolished by such. Thus, we showed that the heterogeneous environment comprising a distinct non-fibrillar BM and an adjacent fibrillar ECM evoked a complex invasive phenotype that differed from any homogeneous ECM condition tested and that the described model represents a physiologically highly relevant setting for addressing cellular characteristics and treatment responses in metastasizing solid tumors.

### Example 4

Preparing human tumor samples, such as the breast tumor samples used for the present studies involves digesting tumors to separate cancerous and non-cancerous cells and re-combining the cancer cells into a mass termed an organoid. In a variation of work described previously for breast cancer cell lines, a procedure that allows the cancer cells to be surrounded by a thin layer of cell-assembled basement membrane is described. This creates organoids that recapitulate a carcinoma in situ, before a cancer has breached the basement membrane and invaded locally as a prerequisite for metastasis. These studies present the use of this system as a functional assay for invasive capacity in early stage cancers, such as breast cancers, as well as a platform on which to test patient specific therapeutic responses.

### Sample Digestion

Human breast tumor samples were obtained the same day as surgery from the pathology department at the Columbia Medical Campus. Samples were kept at 4° C. in RPMI medium until processing. The tumor section was washed several times with a PBS antibiotic solution (1× penicillin-streptomycin-amphotericin and 1 mg/mL gentam icin).

The general protocol used to process tumor sections into cells is described in DeRose et al 2013. Briefly, the necrotic tissue was cut away from the tumor and the mass was determined (typical mass range 100 mg-600 mg). The sample was minced into roughly 2 mm×4 mm size pieces, washed once more with the antibiotic/phosphate buffered saline solution, and then placed in a digestion buffer containing hyaluronidase and type III collagenase. Digestion was allowed to proceed for approximately 24 hours at 37° C. under gentle mixing conditions to yield a cell suspension. The cell suspension was passed through a 100 μm cell strainer to remove any undigested material. The resulting cell suspension contained a mixture of tumor epithelial cells and stromal cells. The two different populations were separated via differential centrifugation, given the propensity of tumor epithelium to form aggregates and the stromal component to remain as single cells under the digestion conditions. Typically, 5-6 rounds of centrifugation were carried out. The epithelial fraction, termed the “organoid fraction,” and stromal cell fraction, termed the “single cell fraction,” were either frozen or placed directly into tissue culture plates. Cells were cultured in M87 medium according to the protocol (DMEM/F12 supplemented with 2% FBS, 1× insulin-transferrin-selenium, 1× penicillin-streptomycin-glutamine, 5 ng/mL EGF, 0.3 μg/mL hydrocortisone, 0.5 ng/mL cholera toxin, 5 nM 3,3′,5-Triiodo-L-thyronine, 0.5 nM β-estradiol, 5 μM isoproterenol hydrochloride, 50 μM ethanolamine, and 50 μM O-Phosphorlyethanolam ine).

### Organoid Generation

To generate bare organoids, cells from the cancer or single cell fractions were detached from tissue culture plates using Accutase. The cells were added to a cold solution of medium containing 0.2575 mg/mL basement membrane extract (BME) at 4° C. such that the final solution contained 1×10⁵ cells/mL. This cell suspension was added to a low adhesion 96-well plate on ice and centrifuged at 1000×G for 10 minutes at 4° C. After centrifugation, organoids were placed in an incubator at 37° C. and 5% CO₂ and allowed to compact for 24 hours. The organoids were deshelled in cell recovery solution for 1 hour prior to implantation in hydrogels.

To generate organoids with a homogeneous shell, the same centrifugation conditions described above were employed except the concentration of BME was changed to 0.25 mg/mL and collagen IV was added at concentrations ranging from 0.003 mg/mL and 0.0075 mg/mL (as also described in the recently submitted manuscript dealing with shell formation for spheroids prepared from cell lines (Guzman et al in revision)). Both fluorescently labeled and unlabeled collagen IV were used for experiments. Prior to implantation in hydrogels, spheroids were briefly washed in warm PBS to remove any debris and/or excess collagen IV.

### Implantation in Hydrogels

Pepsin-treated (PT) collagen I gels were used for organoid implantation. Collagen I gels were made by mixing 10% (v/v) 10× DMEM, 2.5% (v/v) 1 M HEPES, 2.5% (v/v) 7.5% Sodium Bicarbonate, and the necessary amount of collagen I stock (typically around 6 mg/mL) and deionized water to achieve a 1 mg/mL collagen I solution. 1 N NaOH was added to bring the pH of the solution to 7.4 after mixing all other components. Shortly after preparing this solution, 200 μL of this solution was pipetted into 10 mm glass cylinders glued to a glass bottom dish. The multicellular spheroid (MTS) was added to the solution in a 5 μL portion and the dish was incubated at 37° C. for approximately 1 hour to allow gelation to occur. After gelation, gels were overlaid with 50 μL medium, and 1 mL of medium was placed around the cylinder for humidification.

### Results

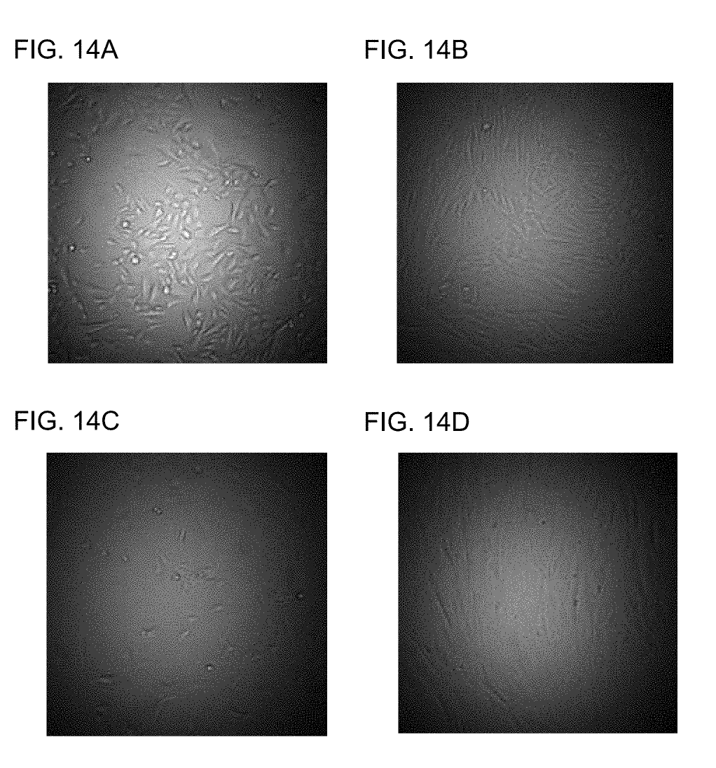

Cells from two different patient samples were plated on substrates to demonstrate differences between those from the cancer cell and non-cancer cell fractions. FIG. 14A-FIG. 14D show that the organoid fractions display epithelial morphology while the single cell fraction is fibroblast like.

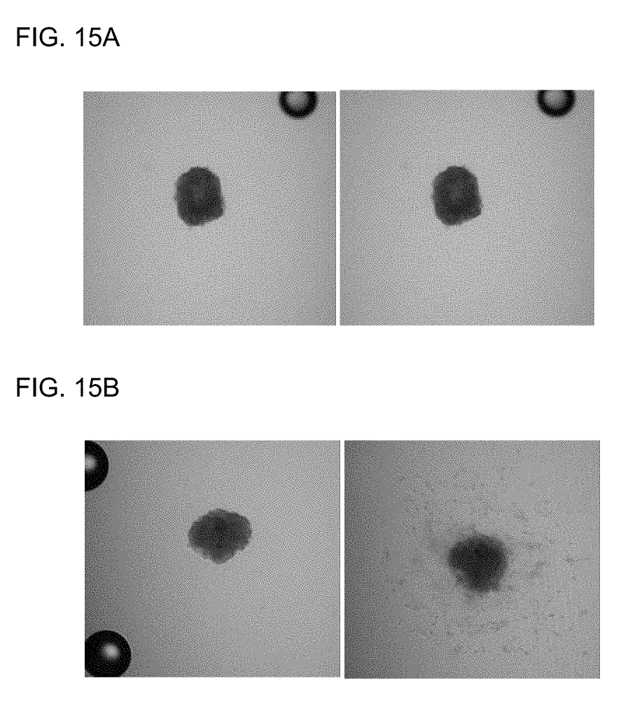

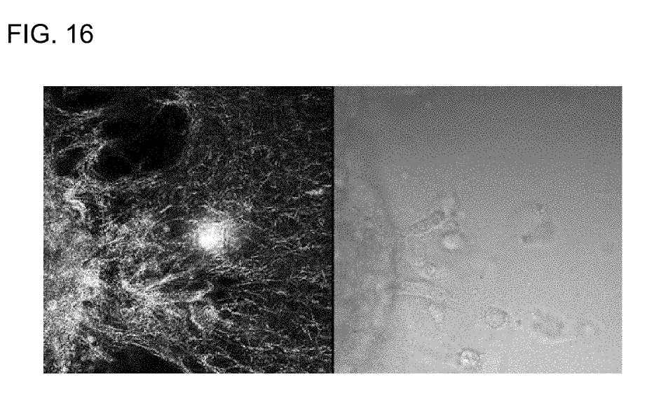

Organoids prepared from cancer and non-cancerous cell fractions were implanted in 1 mg/mL collagen I gels to assess their invasive capacity. This was first done with bare organoids with no basement membrane shell. No invasion was observed. This is representative of organoids formed from the non-cancerous single cell fraction (FIG. 15A). In contrast to that behavior, bare organoids prepared from the cancerous cell fraction often exhibited invasion after 24 hours in the gel (FIG. 15B). In invading spheroids, evidence of matrix reorganization was clear as displayed by collagen fibril alignment in confocal reflectance images (FIG. 16).

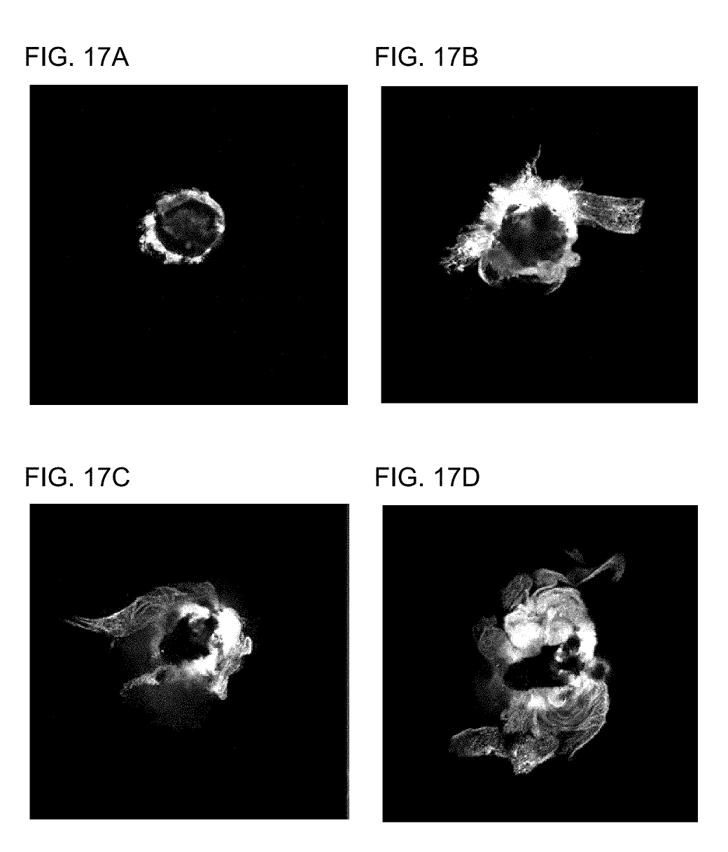

To prepare shelled organoids, collagen IV and BME were used during centrifugation as described above. While BME centrifuged spheroids form a basement membrane shell that is removed in the deshelling step in cell recovery solution, this shell is variable in thickness, composition, and is often incomplete. The use of collagen IV in combination with BME leads to a shell that is continuous, controllable in size (Guzman et al in revision), and often symmetrical. The symmetry of the shell is greatest when using relatively low concentrations of collagen IV. A collagen IV concentration of 0.003 mg/mL was used as a lower limit to observe shell formation and 0.0075 mg/mL was used as an upper limit. Fluorescently labeled collagen IV was used to visualize. Collagen IV shells surrounding organoids for organoids formed from non-cancerous cell fractions at collagen IV concentrations of 0.003 mg/mL and 0.0075 mg/mL as shown in FIG. 17A-FIG. 17B. Shell formation also occurred for the cancerous organoid fractions was similar (FIG. 17C-FIG. 17D).

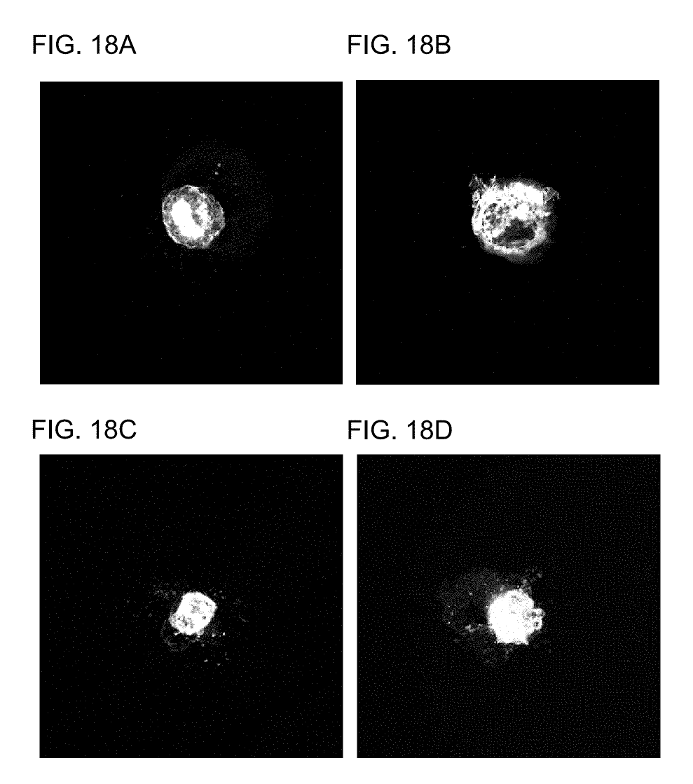

Some heterogeneity in shell size and shape existed across different samples. For example, compare organoids prepared from non-cancerous single cell fractions (FIG. 18A-FIG. 18B) and cancer cell fractions (FIG. 18C-FIG. 18D) in different samples. Cells from these tumors were less efficient in shell assembly than the cells from the tumors shown in FIG. 17A-FIG. 17D.

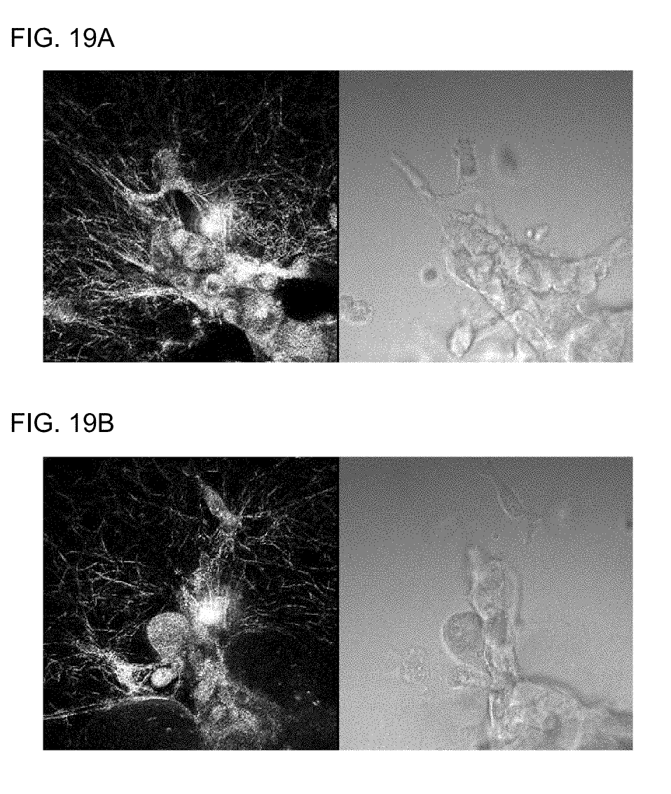

Invasion was observed in shelled organoids prepared from cancerous cell fractions. Invasion is shown from two such organoids prepared from one sample, each with a 0.0075 mg/mL collagen IV concentration (FIG. 19A-FIG.19B). The collagen alignment, primarily multicellular streaming style invasion, and presence of some single cells following breaching of the shell are all consistent with what has been seen in breast cancer cell lines prepared as shelled spheroids as well as in intravital imaging in patients (Guzman et al in revision, Provenzano et al 2006).

### REFERENCES

All patents, patent applications, and publications cited below are incorporated herein by reference in their entirety as if recited in full herein.
1. Hay, M., et al., Clinical development success rates for investigational drugs. Nat Biotechnol, 2014. 32(1): p. 40-51.
2. Breslin, S. and L. O'Driscoll, Three-dimensional cell culture: the missing link in drug discovery. Drug Discov Today, 2013. 18(5-6): p. 240-9.
3. Kimlin, L. C., G. Casagrande, and V.M.Virador, In vitro three-dimensional (3D) models in cancer research: an update. Mol Carcinog, 2013. 52(3): p. 167-82.
4. Kelley, L. C., et al., Traversing the basement membrane in vivo: a diversity of strategies. J Cell Biol, 2014. 204(3): p. 291-302.
5. Linde, N., et al., Integrating macrophages into organotypic co-cultures: a 3D in vitro model to study tumor-associated macrophages. PLoS One, 2012. 7(7): p. e40058.
6. Pickup, M. W., J. K. Mouw, and V. M. Weaver, The extracellular matrix modulates the hallmarks of cancer. EMBO Rep, 2014. 15(12): p. 1243-53.
7. Condeelis, J. and J. W. Pollard, Macrophages: obligate partners for tumor cell migration, invasion, and metastasis. Cell, 2006. 124(2): p. 263-6.
8. Cardoso, A. P., et al., Macrophages stimulate gastric and colorectal cancer invasion through EGFR Y(1086), c-Src, Erk1/2 and Akt phosphorylation and smalIGTPase activity. Oncogene, 2014. 33(16): p. 2123-33.
9. Yang, Y. L., L. M. Leone, and L. J. Kaufman. “Elastic moduli of collagen gels can be predicted from two dimensional confocal microscopy,” Biophys. J, 97, 2051-2060 (2009). PMCID: PMC2756380.
10. Valastyan S, Weinberg RA. Tumor metastasis: molecular insights and evolving paradigms. Cell. 2011;147:275-92.
11. Kumar S, Weaver V M. Mechanics, malignancy, and metastasis: the force journey of a tumor cell. Cancer metastasis reviews. 2009;28:113-27.
12. Lu P, Weaver V M, Werb Z. The extracellular matrix: a dynamic niche in cancer progression. The Journal of cell biology. 2012;196:395-406.
13. Kalluri R. Basement membranes: structure, assembly and role in tumour angiogenesis. Nature reviews Cancer. 2003;3:422-33.
14. Yurchenco P D. Basement membranes: cell scaffoldings and signaling platforms. Cold Spring Harbor perspectives in biology. 2011;3.
15. Liotta L A, Tryggvason K, Garbisa S, Hart I, Foltz C M, Shafie S. Metastatic potential correlates with enzymatic degradation of basement membrane collagen. Nature. 1980;284:67-8.
16. Martinez-Hernandez A, Amenta P S. The basement membrane in pathology. Laboratory investigation; a journal of technical methods and pathology. 1983;48:656-77.
17. Frei J V. The fine structure of the basement membrane in epidermal tumors. The Journal of cell biology. 1962;15:335-42.
18. Bosman F T, Havenith M, Cleutjens J P. Basement membranes in cancer. Ultrastructural pathology. 1985;8:291-304.
19. Spaderna S, Schmalhofer O, Hlubek F, Berx G, Eger A, Merkel S, et al. A transient, EMT-linked loss of basement membranes indicates metastasis and poor survival in colorectal cancer. Gastroenterology. 2006;131:830-40.
20. Bergamaschi A, Tagliabue E, Sorlie T, Naume B, Triulzi T, Orlandi R, et al. Extracellular matrix signature identifies breast cancer subgroups with different clinical outcome. The Journal of pathology. 2008;214:357-67.
21. Polyak K. Molecular markers for the diagnosis and management of ductal carcinoma in situ. Journal of the National Cancer Institute Monographs. 2010;2010:210-3.
22. Mouw J K, Ou G, Weaver V M. Extracellular matrix assembly: a multiscale deconstruction. Nature reviews Molecular cell biology. 2014;15:771-85.
23. Zhu G G, Risteli L, Makinen M, Risteli J, Kauppila A, Stenback F. Immunohistochemical study of type I collagen and type I pN-collagen in benign and malignant ovarian neoplasms. Cancer. 1995;75:1010-7.
24. Kauppila S, Stenback F, Risteli J, Jukkola A, Risteli L. Aberrant type I and type III collagen gene expression in human breast cancer in vivo. The Journal of pathology. 1998;186:262-8.
25. Huijbers I J, Iravani M, Popov S, Robertson D, Al-Sarraj S, Jones C, et al. A role for fibrillar collagen deposition and the collagen internalization receptor endo180 in glioma invasion. PloS one. 2010;5:e9808.
26. Alowami S, Troup S, Al-Haddad S, Kirkpatrick I, Watson P H. Mammographic density is related to stroma and stromal proteoglycan expression. Breast cancer research : BCR. 2003;5:R129-35.
27. Guo Y P, Martin L J, Hanna W, Banerjee D, Miller N, Fishell E, et al. Growth factors and stromal matrix proteins associated with mammographic densities. Cancer epidemiology, biomarkers & prevention : a publication of the American Association for Cancer Research, cosponsored by the American Society of Preventive Oncology. 2001;10:243-8.
28. Provenzano P P, Eliceiri K W, Campbell J M, Inman D R, White J G, Keely P J. Collagen reorganization at the tumor-stromal interface facilitates local invasion. BMC medicine. 2006;4:38.
29. Conklin M W, Eickhoff J C, Riching K M, Pehlke C A, Eliceiri K W, Provenzano P P, et al. Aligned collagen is a prognostic signature for survival in human breast carcinoma. The American journal of pathology. 2011;178:1221-32.
30. Kalluri R, Weinberg R A. The basics of epithelial-mesenchymal transition. The Journal of clinical investigation. 2009;119:1420-8.
31. Gurzu S, Turdean S, Kovecsi A, Contac A O, Jung I. Epithelial-mesenchymal, mesenchymal-epithelial, and endothelial-mesenchymal transitions in malignant tumors: An update. World journal of clinical cases. 2015;3:393-404.
32. Leight J L, Wozniak M A, Chen S, Lynch M L, Chen C S. Matrix rigidity regulates a switch between TGF-beta1-induced apoptosis and epithelial-mesenchymal transition. Molecular biology of the cell. 2012;23:781-91.
33. Wei S C, Fattet L, Tsai J H, Guo Y, Pai V H, Majeski H E, et al. Matrix stiffness drives epithelial-mesenchymal transition and tumour metastasis through a TWIST1-G3BP2 mechanotransduction pathway. Nature cell biology. 2015;17:678-88.
34. Levental K R, Yu H, Kass L, Lakins J N, Egeblad M, Erler J T, et al. Matrix crosslinking forces tumor progression by enhancing integrin signaling. Cell. 2009;139:891-906.
35. Lang N R, Skodzek K, Hurst S, Mainka A, Steinwachs J, Schneider J, et al. Biphasic response of cell invasion to matrix stiffness in three-dimensional biopolymer networks. Acta biomaterialia. 2015;13:61-7.
36. Guzman A, Ziperstein M J, Kaufman L J. The effect of fibrillar matrix architecture on tumor cell invasion of physically challenging environments. Biomaterials. 2014; 35:6954-63.
37. Condeelis J, Segall J E. Intravital imaging of cell movement in tumours. Nature reviews Cancer. 2003;3:921-30.
38. Ellenbroek S I, van Rheenen J. Imaging hallmarks of cancer in living mice. Nature reviews Cancer. 2014;14:406-18.
39. Thoma C R, Zimmermann M, Agarkova I, Kelm J M, Krek W. 3D cell culture systems modeling tumor growth determinants in cancer target discovery. Advanced drug delivery reviews. 2014;69-70:29-41.
40. Katz E, Dubois-Marshall S, Sims A H, Gautier P, Caldwell H, Meehan R R, et al. An in vitro model that recapitulates the epithelial to mesenchymal transition (EMT) in human breast cancer. PloS one. 2011;6:e17083.
41. Schoumacher M, Goldman R D, Louvard D, Vignjevic D M. Actin, microtubules, and vimentin intermediate filaments cooperate for elongation of invadopodia. The Journal of cell biology. 2010;189:541-56.
42. Ivascu A, Kubbies M. Rapid generation of single-tumor spheroids for high-throughput cell function and toxicity analysis. Journal of biomolecular screening. 2006;11:922-32.
43. Wolf K, Mazo I, Leung H, Engelke K, von Andrian U H, Deryugina E l, et al. Compensation mechanism in tumor cell migration: mesenchymal-amoeboid transition after blocking of pericellular proteolysis. The Journal of cell biology. 2003;160:267-77.
44. Fay M P, Proschan M A. Wilcoxon-Mann-Whitney or t-test? On assumptions for hypothesis tests and multiple interpretations of decision rules. Statistics surveys. 2010;4:1-39.
45. Ziperstein M J, Guzman A, Kaufman L J. Breast Cancer Cell Line Aggregate Morphology Does Not Predict Invasive Capacity. PloS one. 2015;10:e0139523.
46. Hanahan D, Weinberg R A. The hallmarks of cancer. Cell. 2000;100:57-70.
47. Rowe R G, Weiss S J. Breaching the basement membrane: who, when and how? Trends in cell biology. 2008;18:560-74.
48. Bosman F T. The borderline: basement membranes and the transition from premalignant to malignant neoplasia. Microscopy research and technique. 1994;28:216-25.
49. Raghavan S, Bauer C, Mundschau G, Li Q, Fuchs E. Conditional ablation of betal integrin in skin. Severe defects in epidermal proliferation, basement membrane formation, and hair follicle invagination. The Journal of cell biology. 2000;150:1149-60.
50. McKee K K, Harrison D, Capizzi S, Yurchenco P D. Role of laminin terminal globular domains in basement membrane assembly. The Journal of biological chemistry. 2007;282:21437-47.
51. Li S, Liquari P, McKee K K, Harrison D, Patel R, Lee S, et al. Laminin-sulfatide binding initiates basement membrane assembly and enables receptor signaling in Schwann cells and fibroblasts. The Journal of cell biology. 2005;169:179-89.
52. Soofi S S, Last J A, Liliensiek S J, Nealey P F, Murphy C J. The elastic modulus of Matrigel as determined by atomic force microscopy. Journal of structural biology. 2009;167:216-9.
53. Halfter W, Oertle P, Monnier C A, Camenzind L, Reyes-Lua M, Hu H, et al. New concepts in basement membrane biology. The FEBS journal. 2015;282:4466-79.
54. Even-Ram S, Yamada K M. Cell migration in 3D matrix. Current opinion in cell biology. 2005;17:524-32.
55. Notary K, Li X Y, Allen E, Stevens S L, Weiss S J. A cancer cell metalloprotease triad regulates the basement membrane transmigration program. Genes & development. 2006;20:2673-86.
56. Sodek K L, Brown T J, Ringuette M J. Collagen I but not Matrigel matrices provide an MMP-dependent barrier to ovarian cancer cell penetration. BMC cancer. 2008;8:223.
57. Wolf K, Te Lindert M, Krause M, Alexander S, Te Riet J, Willis A L, et al. Physical limits of cell migration: control by ECM space and nuclear deformation and tuning by proteolysis and traction force. The Journal of cell biology. 2013;201:1069-84.
58. Zaman M H, Trapani L M, Sieminski A L, Mackellar D, Gong H, Kamm R D, et al. Migration of tumor cells in 3D matrices is governed by matrix stiffness along with cell-matrix adhesion and proteolysis. Proceedings of the National Academy of Sciences of the United States of America. 2006;103:10889-94.
59. Petrie R J, Gavara N, Chadwick R S, Yamada K M. Nonpolarized signaling reveals two distinct modes of 3D cell migration. The Journal of cell biology. 2012;197:439-55.
60. Hanahan D, Weinberg R A. Hallmarks of cancer: the next generation. Cell. 2011;144:646-74.
61. Ohike N, Coban I, Kim G E, Basturk O, Tajiri T, Krasinskas A, et al. Tumor budding as a strong prognostic indicator in invasive ampullary adenocarcinomas. The American journal of surgical pathology. 2010;34:1417-24.
62. Mitrovic B, Schaeffer D F, Riddell R H, Kirsch R. Tumor budding in colorectal carcinoma: time to take notice. Modern pathology: an official journal of the United States and Canadian Academy of Pathology, Inc. 2012;25:1315-25.
63. Karamitopoulou E, Zlobec I, Gloor B, Kondi-Pafiti A, Lugli A, Perren A. Loss of Raf-1 kinase inhibitor protein (RKIP) is strongly associated with high-grade tumor budding and correlates with an aggressive phenotype in pancreatic ductal adenocarcinoma (PDAC). Journal of translational medicine. 2013;11:311.
64. Liang F, Cao W, Wang Y, Li L, Zhang G, Wang Z. The prognostic value of tumor budding in invasive breast cancer. Pathology, research and practice. 2013;209:269-75.
65. Sun Y, Liang F, Cao W, Wang K, He J, Wang H, et al. Prognostic value of poorly differentiated clusters in invasive breast cancer. World journal of surgical oncology. 2014;12:310.
66. Das T, Safferling K, Rausch S, Grabe N, Boehm H, Spatz J P. A molecular mechanotransduction pathway regulates collective migration of epithelial cells. Nature cell biology. 2015;17:276-87.
67. Graves M L, Cipollone J A, Austin P, Bell E M, Nielsen J S, Gilks C B, et al. The cell surface mucin podocalyxin regulates collective breast tumor budding. Breast cancer research : BCR. 2016;18:11.
68. Kaufman L J, Brangwynne C P, Kasza K E, Filippidi E, Gordon V D, Deisboeck T S, et al. Glioma expansion in collagen I matrices: analyzing collagen concentration-dependent growth and motility patterns. Biophys J. 2005;89:635-50.
69. Yang Y L, Motte S, Kaufman L J. Pore size variable type I collagen gels and their interaction with glioma cells. Biomaterials. 2010;31:5678-88.
70. Nguyen-Ngoc K V, Cheung K J, Brenot A, Shamir E R, Gray R S, Hines W C, et al. ECM microenvironment regulates collective migration and local dissemination in normal and malignant mammary epithelium. Proceedings of the National Academy of Sciences of the United States of America. 2012;109:E2595-604.
71. Harma V, Virtanen J, Makela R, Happonen A, Mpindi J P, Knuuttila M, et al. A comprehensive panel of three-dimensional models for studies of prostate cancer growth, invasion and drug responses. PloS one. 2010;5:e10431.
72. Coussens L M, Fingleton B, Matrisian L M. Matrix metalloproteinase inhibitors and cancer: trials and tribulations. Science. 2002;295:2387-92.
73. Tse J M, Cheng G, Tyrrell JA, Wilcox-Adelman S A, Boucher Y, Jain R K, et al. Mechanical compression drives cancer cells toward invasive phenotype. Proceedings of the National Academy of Sciences of the United States of America. 2012;109:911-6.
74. Milosevic M, Fyles A, Hedley D, Pintilie M, Levin W, Manchul L, et al. Interstitial fluid pressure predicts survival in patients with cervix cancer independent of clinical prognostic factors and tumor oxygen measurements. Cancer research. 2001;61:6400-5.
75. Hompland T, Ellingsen C, Ovrebo K M, Rofstad E K. Interstitial fluid pressure and associated lymph node metastasis revealed in tumors by dynamic contrast-enhanced MRI. Cancer research. 2012;72:4899-908.
76. Polacheck W J, German A E, Mammoto A, Ingber D E, Kamm R D. Mechanotransduction of fluid stresses governs 3D cell migration. Proceedings of the National Academy of Sciences of the United States of America. 2014;111:2447-52.
77. DeRose et. al. Patient-derived Models of Human Breast Cancer: Protocols for In vitro and In vivo Applications in Tumor Biology and Translational Medicine. Curr Protoc Pharmacol. 2013.
78. Guzman A., Alemany V. S., Nguyen Y., Zhang C. R., Kaufman L .J. A novel 3D in vitro metastasis model elucidates differential invasive strategies during and after breaching basement membrane. Biomaterials. In Revision.

What is claimed is:

1. An in vitro system for evaluating a therapeutic response to a candidate therapeutic agent, the system comprising:
(i) a multicellular aggregate;
(ii) a cell-bound layer of basement membrane surrounding the multicellular aggregate; and
(iii) a three-dimensional (3-D) biopolymer matrix,
wherein the multicellular aggregate and the cell-bound layer of basement membrane are disposed within the 3-D biopolymer matrix.

2. The in vitro system of claim 1, wherein the 3-D biopolymer matrix is a biopolymer solution comprising collagen I, collagen IV, basement membrane extract (BME), or a combination thereof, that undergoes gelation.

3. The in vitro system of claim 1, which is a high throughput system.

4. A method of preparing an in vitro system for evaluating a therapeutic response to a candidate therapeutic agent, the method comprising:
(a) suspending cells in a growth medium supplemented with a basement membrane extract;
(b) centrifuging the suspended cells, followed by incubating the cells under conditions sufficient to form a multicellular aggregate surrounded by a layer of basement membrane; and
(c) disposing the multicellular aggregate surrounded by the layer of basement membrane in a 3-D extracellular matrix.

5. The method of claim 4, wherein the 3-D extracellular matrix is a biopolymer comprising collagen I, collagen IV, basement membrane extract (BME), or a combination thereof.

6. The method of claim 4, wherein the basement membrane is assembled by the cells.

7. A method for evaluating a therapeutic response to a candidate therapeutic agent in an in vitro system, the method comprising:
(a) providing a candidate therapeutic agent to the in vitro system according to claim 1; and
(b) evaluating the response of cells in the biopolymer matrix to the candidate therapeutic agent.

8. A method for identifying a candidate therapeutic agent as a candidate anti-cancer drug, the method comprising:
(a) contacting a candidate therapeutic agent with the in vitro system according to claim 1; and
(b) evaluating what effect, if any, the candidate therapeutic agent has on the in vitro system,
wherein decreased migratory capacities and/or increased cell death of the cells in the multicellular aggregate relative to a control indicates that the candidate therapeutic agent may be a candidate anti-cancer drug.

9. A method for diagnosing the presence of tumorigenic cells in a subject, the method comprising:
(a) obtaining cells from the subject;
(b) incubating the cells under conditions sufficient to form a multicellular aggregate surrounded by a layer of basement membrane;
(c) disposing the multicellular aggregate surrounded by the layer of basement membrane in a 3-D extracellular matrix;
(d) culturing the 3-D extracellular matrix under conditions sufficient to support growth of the cells; and
(e) identifying the cells as tumorigenic cells if the multicellular aggregate breaches the layer of basement membrane into the 3-D extracellular matrix.

10. The method of claim 9, wherein the cells obtained from the subject are primary tumor cells.

11. The method of claim 9, wherein the 3-D extracellular matrix is a biopolymer comprising collagen I, collagen IV, basement membrane extract (BME), or a combination thereof.

12. The method of claim 9, wherein the basement membrane is assembled by the cells.

13. The method of claim 9, wherein the subject is a mammal.

14. The method of claim 13, wherein the mammal is selected from the group consisting of humans, primates, farm animals, and domestic animals.

15. The method of claim 13, wherein the mammal is a human.

16. The method of claim 9, wherein the tumorigenic cells are carcinoma cells.

17. The method of claim 9, wherein the tumorigenic cells are breast cancer cells.

18. A method of treating or ameliorating the effects of a cancer in a subject, the method comprising:
(a) diagnosing the presence of tumorigenic cells in the subject by the method of claim 9; and
(b) administering to the subject an effective amount of an anti-cancer drug.

19. The method of claim 18, wherein the anti-cancer drug is determined according to the method of claim 8.

20. The method of claim 18, wherein the subject is a mammal.

21. The method of claim 20, wherein the mammal is selected from the group consisting of humans, primates, farm animals, and domestic animals.

22. The method of claim 20, wherein the mammal is a human.

23. The method of claim 18, wherein the cancer is a carcinoma.

24. The method of claim 18, wherein the cancer is breast cancer.
